SEQUENCE LISTING (18 sequences)

SEQ ID NO: 1 - protein, 18 aa, Artificial Sequence
  integrin-binding site within human neuregulin 1 EGF-like region
VKCAEKEKTFCVNGGECF

SEQ ID NO: 2 - protein, 45 aa, Artificial Sequence
  integrin-binding site within human neuregulin 1 EGF-like region and portion of alpha domain
HLVKCAEKEKTFCVNGGECFMVKDLSNPSRYLCKCQPGFTGARCT

SEQ ID NO: 3 - protein, 48 aa, Artificial Sequence
  integrin-binding site within human neuregulin 1 EGF-like region and portion of alpha domain, residues 175-222 of human neuregulin 1 alpha
GTSHLVKCAEKEKTFCVNGGECFMVKDLSNPSRYLCKCQPGFTGARCT

SEQ ID NO: 4 - protein, 640 aa, Homo sapiens
  human neuregulin 1alpha (NRG1alpha)
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 5 - protein, 67 aa, Artificial Sequence
  integrin-binding site within human neuregulin, residues 175-241of human neuregulin 1alpha, long version
GTSHLVKCAEKEKTFCVNGGECFMVKDLSNPSRYLCKCQPGFTGARCTENVPMKVQNQEKAEELYQK

SEQ ID NO: 6 - protein, 67 aa, Artificial Sequence
  integrin-binding site within human neuregulin, residues 175-241of human neuregulin 1alpha, long version, 3KE mutant
GTSHLVECAEEEETFCVNGGECFMVKDLSNPSRYLCKCQPGFTGARCTENVPMKVQNQEKAEELYQK

SEQ ID NO: 7 - protein, 67 aa, Artificial Sequence
  integrin-binding site within human neuregulin, residues 175-241of human neuregulin 1alpha, long version, 2KE mutant
GTSHLVKCAEEEETFCVNGGECFMVKDLSNPSRYLCKCQPGFTGARCTENVPMKVQNQEKAEELYQK

SEQ ID NO: 8 - protein, 645 aa, Homo sapiens
  human neuregulin 1beta (NRG1beta)
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 9 - protein, 72 aa, Artificial Sequence
  human neuregulin 1beta long, residues 175-246 of human neuregulin 1beta, long version
GTSHLVKCAEKEKTFCVNGGECFMVKDLSNPSRYLCKCPNEFTGDRCQNYVMASFYKHLGIEFMEAEELYQK

SEQ ID NO: 10 - protein, 71 aa, Artificial Sequence
  murine neuregulin 1beta fragment
TSHLIKCAEKEKTFCVNGGECFMVKDLSNPSRYLCKCPNEFTGDRCQNYVMASFYKHLGIEFMEAEELYQK

SEQ ID NO: 11 - protein, 48 aa, Artificial Sequence
  integrin-binding site within human neuregulin, 3KE mutant, short version
GTSHLVECAEEEETFCVNGGECFMVKDLSNPSRYLCKCQPGFTGARCT

SEQ ID NO: 12 - protein, 48 aa, Artificial Sequence
  integrin-binding site within human neuregulin, 2KE mutant, short version
GTSHLVKCAEEEETFCVNGGECFMVKDLSNPSRYLCKCQPGFTGARCT

SEQ ID NO: 13 - protein, 52 aa, Artificial Sequence
  neuregulin 1alpha (NRG-1alpha) EGF-like region integrin-binding site
HLVKCAEKEKTFCVNGGECFMVKDLSNPSRYLCKCQPGFTGARCTENVPMKV

SEQ ID NO: 14 - protein, 52 aa, Artificial Sequence
  neuregulin 1beta (NRG-1beta) EGF-like region integrin-binding site
HLVKCAEKEKTFCVNGGECFMVKDLSNPSRYLCKCPNEFTGDRCQNYVMASF

SEQ ID NO: 15 - protein, 49 aa, Artificial Sequence
  neuregulin 2alpha (NRG-2alpha) EGF-like region integrin-binding site
HARKCNETAKSYCVNGGVCYYIEGINQLSCKCPNGFFGQRCLEKLPLRL

SEQ ID NO: 16 - protein, 49 aa, Artificial Sequence
  neuregulin 2beta (NRG-2beta) EGF-like region integrin-binding site
HARKCNETAKSYCVNGGVCYYIEGINQLSCKCPVGYTGDRCQQFAMVNF

SEQ ID NO: 17 - protein, 51 aa, Artificial Sequence
  neuregulin 3 (NRG-3) EGF-like region integrin-binding site
HFKPCRDKDLAYCLNDGECFVIETLTGSHKHCRCKEGYQGVRCDQFLPKTD

SEQ ID NO: 18 - protein, 49 aa, Artificial Sequence
  neuregulin 4 (NRG-4) EGF-like region integrin-binding site
HEQPCGPRHRSFCLNGGICYVIPTIPSPFCRCIENYTGARCEEVFLPSS

(19) United States
(12) Patent Application Publication
(10) Pub. No.: US 2011/0212108 A1
(43) Pub. Date: Sep. 1, 2011

(54) NEUREGULIN/ERBB SIGNALING AND INTEGRIN

(75) Inventor: Yoshikazu Takada, Davis, CA (US)

(73) Assignee: THE REGENTS OF THE UNIVERSITY OF CALIFORNIA, OAKLAND, CA (US)

(21) Appl. No.: 12/991,908

(22) PCT Filed: May 11, 2009
(86) PCT No.: PCT/US09/43473

§ 371 Date: May 9, 2011

Related U.S. Application Data
(60) Provisional application No. 61/051,961, filed on May 9, 2008.

(51) Int. Cl. A61K 39/395; G01N 33/68; C12N 5/071; C12N 5/10; C12N 1/00; C07K 14/47; A61K 38/17; C12N 15/79; C07H 21/00; C12N 15/63; A61K 31/7088; A61P 35/00; A61K 38/12; A61K 38/45

(52) U.S. Cl. 424/172.1; 435/7.21; 435/375; 435/325; 435/419; 435/243; 436/501; 530/324; 514/21.3; 435/455; 536/23.5; 435/320.1; 514/44 R; 435/7.1; 514/19.3; 514/19.9; 424/94.5

(57) ABSTRACT

The present invention resides in the discovery that the specific interaction between neuregulin 1 (NRG1) and integrin is important for ErbB signaling, which in turn plays an important role in cellular signaling in various physiological processes such as cell proliferation, especially in cancer cells overexpressing ErbB family members. Thus, this invention provides for a novel method for inhibiting ErbB signaling by using an inhibitor of NRG1-integrin binding. A method for identifying inhibitors of NRG1-integrin binding is also described. Further disclosed are polypeptides, nucleic acids, and corresponding compositions for inhibiting ErbB signaling.

### RELATED APPLICATIONS

This application claims priority to U.S. Provisional Patent Application No. 61/051,961, filed May 9, 2008, the contents of which are incorporated by reference in the entirety.

### STATEMENT AS TO RIGHTS TO INVENTIONS MADE UNDER FEDERALLY SPONSORED RESEARCH AND DEVELOPMENT

This invention was made with Government support under Grant No. AG027350 by the National Institutes of Health. The Government has certain rights in this invention.

### BACKGROUND OF THE INVENTION

The neuregulins (NRGs) are a family of four structurally related proteins that are part of the epidermal growth factor (EGF) family of proteins. They contain an EGF-like motif that binds to and activates receptor tyrosine kinases in the EGF receptor (ErbBs) family (ErbB3 and ErbB4). Neuregulin-1 (NRG1) plays essential roles in the nervous system, heart, and breast. Over 15 distinct isoforms of neuregulin-1 have been identified. Neuregulin-1 isoforms can be divided into two large groups, known as α- and β-types, based on structural differences in their EGF binding domains (Holmes, Sliwkowski et al. 1992). NRG1 signaling is involved in the development and functions of several other organ systems, and human diseases, including schizophrenia, coronary heart diseases, and cancer (Falls 2003). Targeted deletion of ErbB2, ErbB3, ErbB4, or NRG1 in mice leads to developmental abnormalities that are severe in the nervous system and lethal in the cardiovascular system (Falls 2003). In cancers, the interaction between ErbB receptors and ligands such as NRGs plays an important role in tumor growth. The EGF-like motif of NRGs is essential and sufficient for receptor binding and activation as well as promoting tumorigenesis (Breuleux 2007). The presence of autocrine loop is what induces aberrant ErbB receptor activation, and has been correlated with cancer development and progression. Disrupting this autocrine loop may provide an important therapeutic measure to control cancer cell growth (Li et al. 2004).

The EGF receptor (EGFR, ErbBs) family consists of ErbB1, ErbB2, ErbB3, and ErbB4, which differ in their ability to bind ligand or elicit a signal. ErbB2 has no direct ligand. In comparison, ErbB3 can bind NRG1, but it is lacking intracellular kinase activity. The ErbB4 receptor has the ability to bind NRG1 and also contains a highly active tyrosine kinase domain (Carraway and Cantley 1994). Once NRG1 is bound, it stimulates homologous and heterologous dimerization of EGF receptor family members, leading to the phosphorylation of tyrosine residues. ErbB3 can dimerize with ErbB2 and ErbB4, but it is the dimerization of ErbB2 with ErbB4 that can form the highest affinity-binding site and greatly enhance the level of tyrosine phosphorylation (Carpenter 2003). In vivo, functional NRG1 receptors are heterodimers composed of ErbB2 with either an ErbB3, or ErbB4 molecule. ErbB2 is a preferred partner of other activated ErbB receptors, as it has a fixed conformation that resembles the ligand-activated state, and thus is permanently poised for interaction with the other activated ErbBs (Hynes and Lane 2005).

Evidence implicates the aberrant activation of ErbB receptors in the progression of various human tumors, notably breast and ovarian cancers (Slamon et al. 1989). Overexpression of EGF receptor, ErbB2, and ErbB3 has been observed in numerous solid tumors types, and correlates with a high degree of receptor activation (Holbro et al. 2003; Roskoski 2004). Amplification of the erbB2 gene is observed in 25-30% of breast cancer patients, and overexpression of the product correlates with earlier relapse and poor prognosis (Slamon et al. 1987; Berger et al. 1988; Slamon et al. 1989). The observed efficacy of the FDA-approved drug trastuzumab (Genentech's Herceptin), a humanized antibody directed to the ErbB2 (HER2/neu) protein, toward ErbB2-positive breast tumors validates this receptor as a therapeutic target (Shak 1999).

Since the aberrant activation of ErbB2 protein tyrosine kinase activity is thought to contribute to tumor progression by engaging specific cellular signaling pathways that promote progression (Kim and Muller 1999), much emphasis has been placed on understanding the biochemical mechanisms by which ErbB2 and its relatives are activated. The members of the ErbB receptor family undergo a network of homo- and heterodimerization events as part of their activation mechanism. Particularly noteworthy is a strong propensity of ErbB2 to heterodimerize with and activate ErbB3, especially when the two receptors are overexpressed (Alroy and Yarden 1997; Riese and Stern 1998; Olayioye et al. 2000). Studies have established a strong link between the coordinate overexpression and activation of ErbB2 and ErbB3 in breast tumor cell lines and in patient samples (Lemoine et al. 1992; Rajkumar and Gullick 1994; Naidu et al. 1998; Siegel et al. 1999). Moreover, in tumors from transgenic mice generated by expressing an active allele of ErbB2, ErbB3 overexpression and activation is also observed (Siegel et al. 1999). On the basis of such expression studies it has been suggested that the ErbB3 receptor may also be used as a marker for patient prognosis (Naidu et al. 1998), and that ErbB3 may contribute to the progression of breast tumor cells from non-invasive to invasive. In vitro, ErbB2 and ErbB3 synergize in promoting the growth and transformation of cultured fibroblasts (Alimandi et al. 1995; Carraway et al. 1995), and numerous studies demonstrate that the two receptors synergize in mediating increased invasiveness induced by NRG1 in breast tumor cell lines (Xu et al. 1997; Tan et al. 1999; Hijazi et al. 2000). Taken together, these observations indicate that there may be an advantage for both receptors to be present and activated in tumor cells to promote breast tumor growth and progression (Siegel et al. 1999; Holbro et al. 2003). It has been reported that ErbB2 and ErbB3 are major EGFRs in human melanoma (Stove et al. 2003), suggesting that the two receptors play important roles in melanoma progression as well.

Integrins have been shown to crosstalk with receptor tyrosine kinase (RTK) in growth factor signaling. Integrins are a family of cell adhesion receptors that recognize extracellular matrix ligands and cell surface ligands (Hynes 2002). Integrins are transmembrane α-β heterodimers, and at least 18α and 8β subunits are known (Shimaoka and Springer 2003). Integrins are involved in signal transduction upon ligand binding, and their functions are in turn regulated by signals from within the cell (Hynes 2002). It has been reported that there is a positive correlation between αvβ3 integrin levels and overexpression of NRG associated with melanoma tumor progression and metastasis (Tang et al. 1996; Atlas et al. 2003; Tsai et al. 2003). It has been proposed that NRG1 may play a key role in the regulation of αvβ3 integrin expression and in its signaling functions (Vellon et al. 2005). The specifics of the role of integrins in NRG1/ErbB signaling were unclear.

It was discovered, in the present invention, that the EGF-like domain of NRG1 directly binds to integrin αvβ3 (and perhaps other integrins). The EGF-like domain of the NRGs is known to be sufficient to specifically activate ErbB receptors and induce cellular responses in culture. The integrin-binding site is located at the N-terminus of the EGF-like domain, which is not involved in ErbB binding according to the crystal structure of the EGF-EGFR complex. It was observed, in the present invention, that wt NRG1 induced ERK1/2 activation in M21 melanoma cells (αvβ3+, ErbB2/ErbB3+, ErbB4−) in vitro, while the integrin-binding-defective mutant of NRG1 (designated 3KE mutant) did not induce cell proliferation, ERK1/2 activation, or AKT activation. Notably, the 3KE mutant suppressed cell proliferation induced by wt NRG1 (a dominant-negative effect by definition) and suppressed ERK1/2 activation (see Preliminary results). It is thus believed that the direct binding of integrins to the EGF-like domain of NRG1 is critical for NRG1/ErbB signaling. It was showed in the present invention that 3KE suppressed the growth of highly metastatic cancer cells in vivo and the growth of pre-malignant legion in vivo. These results indicate that 3KE can be used as a therapeutic agent in treating cancer.

It has been reported that integrins play a critical role in regulating growth factor signaling (e.g., VEGF and IGF-1). In the cases of VEGF (Mahabeleshwar et al. 2007) and IGF-1 (Clemmons et al. 2007) c-Src mediates Tyr-phosphorylation of the β3 tail. In the case of IGF1, the β3 Tyr-phosphorylation induces recruitment of SHP-2 Tyr phosphatase. It was discovered in the present invention that wt NRG1 enhanced the β3 tail phosphorylation in M21 cells. This novel finding supports the belief that the direct integrin binding to NRG1 brings together ErbB receptors and integrins in a physical proximity, and triggers Tyr phosphorylation of the β3 tail. This also facilitates recruitment of other proteins (e.g., SHP-2) to the β3 tail.

It has been well established that the over-expression of ErbB2 and ErbB3 is correlated with the formation of metastatic cancers as described above. In contrast, activation of ErbB4 by NRG either results in proliferation or differentiation (Breuleux 2007). Also expression of ErbB4 receptor is correlated with the incidence of non-metastatic types of human cancers, (Yumoto et al. 2006). Prostate cancers characteristically lack ErbB4 expression while normal prostate luminal cells strongly express ErbB4 (Zamarron et al. 1990). However, the molecular mechanism underlying this phenomenon was unclear.

In the clinic, some trastuzumab (anti-ErbB2)-treated breast cancer patients displayed cardiac phenotypes, including cardiomyopathy, congestive heart failure and decreased left ventricular ejection fraction. It has been proposed that ErbB4 has a role in trastuzumab-induced cardiotoxicity (Hynes and Lane 2005). As described above, ErbB2 has an essential role in the developing heart. Conditional ablation of ErbB2 in postnatal cardiac-muscle cell lineages revealed a role for ErbB2 in the adult heart.

It was showed in the present invention that wt NRG1 enhanced ERK1/2 activation in CHO cells (ErbB2+, ErbB3+, and ErbB4−). Unexpectedly, it was found in the present invention that, in CHO cells that express recombinant human ErbB4 (designated ErbB4-CHO cells), wt NRG1 suppressed ERK1/2 activation, but 3KE enhanced ERK1/2 activation. Thus, 3KE is not defective in ErbB binding. One possibility is that integrins positively regulate ErbB2/ErbB3 signaling, but negatively regulate ErbB2/ErbB4 signaling. This possibility can be tested using the 3KE mutant, which suppressed ErbB3 (as ErbB2/ErbB3) signaling, but did not suppress ErbB4 (as ErbB2/ErbB4) signaling.

It was showed, in the further experiments of the present invention, that 3KE effectively suppressed tumor growth and the outgrowth of pre-cancer lesions in vivo. Although endothelial cells express αvβ3 when they are activated by growth factors (e.g., during angiogenesis), it was unclear which integrins are involved in NRG1 signaling. The evidence of the present invention indicates that keratinocyte integrin α6β4 binds to NRG1. It is believed that α6β4-NRG1 interaction is involved in ErbB2-α6β4 interaction, which has been implicated in cancer progression.

Thus, an NRG1 mutant that does not bind integrin while retaining its ability to bind ErbB (such as the 3KE mutant created in the present invention) has an immediate utility as a therapeutic in cancer. Also, the 3KE mutant is a powerful tool for studying the role of integrins in ErbB signaling. Furthermore, the integrin-binding site within the EGF-like domain of NRG1 provides a valuable tool for identification of additional inhibitors of NRG1-integrin binding, as these inhibitors can be useful in cancer therapy. Because of the prevalence of cancers, there remains a need to develop new strategies for cancer treatment. The present invention addresses this and other related needs.

### BRIEF SUMMARY OF THE INVENTION

The invention is directed to methods and compositions useful for inhibiting proliferation of a cell based on the discovery that the interaction between neuregulin and certain integrin molecules is involved in ErbB-mediated signaling. Therefore, in one aspect of the invention, the present invention provides a method for inhibiting proliferation of a cell, comprising the step of contacting the cell with an effective amount of an inhibitor of neuregulin-integrin binding.

In some embodiments, the neuregulin is neuregulin 1α (NRG1α) or neuregulin 1β (NRG1β). In some embodiments, the integrin is αvβ3, α6β4, α6β1 or α9β1.

In some embodiments, the inhibitor is a polypeptide that inhibits neuregulin-integrin binding, comprising a core amino acid sequence corresponding to residues 197-241 of SEQ ID NO:4 or residues 197-246 of SEQ ID NO:8 or conservative modified variants thereof. In some embodiments, if such polypeptide inhibitor comprises an additional amino acid sequence, e.g., at the N-terminus of the core amino acid sequence, the additional amino acid sequence does not comprise a sequence corresponding to residues 181-187 of SEQ ID NO:4 or 8.

In some embodiments, the inhibitor is a polypeptide that inhibits neuregulin-integrin binding, comprising a core amino acid sequence corresponding to residues 190-241 of SEQ ID NO:4 or residues 190-246 of SEQ ID NO:8 or conservative modified variants thereof. In some embodiments, if such polypeptide inhibitor comprises an additional amino acid sequence, e.g., at the N-terminus of the core amino acid sequence, the additional amino acid sequence does not comprise a sequence corresponding to residues 181-187 of SEQ ID NO:4 or 8.

In some embodiments, the polypeptide inhibitor that inhibits neuregulin-integrin binding comprises the amino acid sequence of residues 175-241 of SEQ ID NO:4 or 175-246 of SEQ ID NO:8 or conservative modified variants thereof, except that at least one of three Lys residues at positions 181, 185, and 187 of SEQ ID NO:4 or SEQ ID NO:8 substituted or deleted. In some embodiments, at least two of the three Lys residues at positions 181, 185, and 187 of SEQ ID NO:4 or SEQ ID NO:8 are substituted. In some embodiments, the inhibitor is a polypeptide comprising the amino acid sequence of SEQ ID NO:6. In some embodiments, the inhibitor is a polypeptide comprising the amino acid sequence of SEQ ID NO:7.

In some embodiments, the polypeptide inhibitor that inhibits neuregulin-integrin binding comprises the amino acid sequence of residues 175-222 of SEQ ID NO:4 or 175-222 of SEQ ID NO:8 or conservative modified variants thereof, with at least one of three Lys residues at positions 181, 185, and 187 of SEQ ID NO:4 or SEQ ID NO:8 substituted or deleted. In some embodiments, at least two of the three Lys residues at positions 181, 185, and 187 of SEQ ID NO:4 or SEQ ID NO:8 are substituted. In some embodiments, the inhibitor is a polypeptide comprising the amino acid sequence GTSHLVECAEEEETFCVNGGECFMVKDLSNPSRYLCKCQPGFTGARCT (SEQ ID NO:11). In some embodiments, the inhibitor is a polypeptide comprising the amino acid sequence GTSHLVKCAEEEETFCVNGGECFMVKDLSNPSRYLCKCQPGFTGARCT (SEQ ID NO:12).

In some embodiments, the polypeptide inhibitor of the present invention is a polypeptide further comprising a heterologous amino acid sequence, e.g., glutathione S-transferase (GST) sequence.

In some embodiments, the inhibitor is anti-β3 antibody 7E3, or cyclic RGDfV. In some embodiments, the cell is within a patient's body. In other embodiments, the contacting step is performed by subcutaneous, intramuscular, intravenous, intraperitoneal, or intratumor injection. In some embodiments, the effective amount of the polypeptide inhibitor of the present invention is 1 μg/kg to 1 mg/kg body weight.

In a second aspect, the present invention relates to a method for identifying an inhibitor of neuregulin-integrin binding. This method comprises the following steps: (1) contacting an integrin and a polypeptide comprising an integrin-binding sequence of a neuregulin or conservative modified variants thereof, in the presence of a test compound, under conditions permissible for neuregulin-integrin binding; and (2) detecting the level of polypeptide-integrin binding, wherein a decrease in the level of binding when compared with the level of binding in the absence of the test compound indicates the compound as an inhibitor of neuregulin-integrin binding.

In some embodiments, the integrin-binding sequence comprises the amino acid sequence of residues 181-187 of SEQ ID NO:4 or 8. In some embodiments, the neuregulin is neuregulin 1α (NRG1α) or neuregulin 1β (NRG1β). In some embodiments, the integrin is αvβ3, α6β4, α6β1 or α9β1. In other embodiments, the polypeptide comprises SEQ ID NO:1, SEQ ID NO:2, SEQ ID NO:3, SEQ ID NO:4, SEQ ID NO:5, SEQ ID NO:8, or SEQ ID NO:9. In some cases, the polypeptide further comprises a heterologous amino acid sequence, for example, a glutathione S-transferase (GST). In some embodiments, the integrin is expressed on a cell surface.

In a third aspect, the present invention relates to an isolated polypeptide inhibits neuregulin-integrin binding, comprising a core amino acid sequence corresponding to residues 197-241 of SEQ ID NO:4 or residues 197-246 of SEQ ID NO:8 or conservative modified variants thereof. In some embodiments, if such isolated polypeptide comprises an additional amino acid sequence, e.g., at the N-terminus of the core amino acid sequence, the additional amino acid sequence does not comprise a sequence corresponding to residues 181-187 of SEQ ID NO:4 or 8. In some embodiments, the core amino acid sequence of the isolated polypeptide corresponds to residues 190-241 of SEQ ID NO:4 or residues 190-246 of SEQ ID NO:8 or conservative modified variants thereof.

In some embodiments, at least two of the three Lys residues at positions 181, 185, and 187 of SEQ ID NO:4 or SEQ ID NO:8 are substituted. In some embodiments, the Lys residues at positions 185 and 187 but not 181 are substituted. In other embodiments, the Lys residues at positions 181, 185, and 187 are substituted. In some cases, each of the Lys residues is substituted with a Glu residue. In some embodiments, the isolated polypeptide is a polypeptide comprising the amino acid sequence of SEQ ID NO:6. In some embodiments, the isolated polypeptide is a polypeptide comprising the amino acid sequence of SEQ ID NO:7. In some embodiments, the isolated polypeptide is a polypeptide comprising the amino acid sequence GTSHLVECAEEEETFCVNGGECFMVKDLSNPSRYLCKCQPGFTGARCT (SEQ ID NO:11). In some embodiments, the isolated polypeptide is a polypeptide comprising the amino acid sequence GTSHLVKCAEEEETFCVNGGECFMVKDLSNPSRYLCKCQPGFTGARCT (SEQ ID NO:12).

The invention also relates to an isolated nucleic acid encoding the polypeptide described herein, as well as a recombinant expression cassette comprising the nucleic acid or an isolated host cell comprising such a recombinant expression cassette.

In a fourth aspect, the present invention relates to a composition comprising (A) a physiologically acceptable excipient and (B) an isolated polypeptide that inhibits neuregulin-integrin binding, comprising a core amino acid sequence corresponding to residues 197-241 of SEQ ID NO:4 or residues 197-246 of SEQ ID NO:8 or conservative modified variants thereof. In some embodiments of the invention, if such isolated polypeptide comprises an additional amino acid sequence, the additional amino acid sequence does not comprise a sequence corresponding to residues 181-187 of SEQ ID NO:4 or 8. In some embodiments of the invention, the isolated polypeptide comprises the amino acid sequence of SEQ ID NO:6 or 7. In some embodiments, the isolated polypeptide is a polypeptide comprising the amino acid sequence GTSHLVECAEEEETFCVNGGECFMVKDLSNPSRYLCKCQPGFTGARCT (SEQ ID NO:11). In some embodiments, the isolated polypeptide is a polypeptide comprising the amino acid sequence GTSHLVKCAEEEETFCVNGGECFMVKDLSNPSRYLCKCQPGFTGARCT (SEQ ID NO:12). In some cases, the polypeptide further comprises a heterologous amino acid sequence, for example, a glutathione S-transferase (GST). The invention also relates to a composition comprising a nucleic acid encoding the polypeptide described herein with a pharmaceutically acceptable excipient.

In a fifth aspect, the present invention relates to a kit for inhibiting neuregulin/ErbB signaling, comprising the composition of a polypeptide or nucleic acid as described herein with a pharmaceutically acceptable excipient. The present invention also relates to a kit for inhibiting proliferation of a cell, comprising the composition of a polypeptide or nucleic acid as described herein with a pharmaceutically acceptable excipient. Instruction manual or user information in other forms is generally included in the kit.

The present invention also relates to a kit for identifying an inhibitor of neuregulin-integrin binding, comprising an integrin and a polypeptide comprising an integrin-binding in-binding sequence of a neuregulin. Optionally, instruction manual or user information in other forms is generally included in the kit.

In a sixth aspect, the present invention relates to a method for inhibiting proliferation of a cell. The method comprises the step of transfecting the cell with a nucleic acid encoding the polypeptide of described herein. In some embodiments, the neuregulin/ErbB signaling in a cell is inhibited using the method described herein.

### BRIEF DESCRIPTION OF THE DRAWINGS

FIG. 1. The wt EGF-like domain of NRG1 (NRG (175-222)) bound to recombinant soluble αvβ3 in ELISA-type assays. The isolated EGF-like domain of Wt NRG1 was immobilized to wells of 96-well titer plates. The concentrations of the coating solution are shown. Soluble recombinant integrin αvβ3 (5 μg/ml) was added to the wells in the presence of 1 mM Mn²⁺ and incubated for 2 h at room temperature. After washing the wells bound αvβ3 was determined by using anti-β3 antibody and HRP-conjugated anti-mouse IgG. The data is shown as means+/−SEM of triplicate experiments.

FIG. 2. Specific adhesion of CHO cells that express human β3 (β3-CHO) to wt NRG (175-222). Adhesion assays were performed as described. mAb 7E3 (to human β3) and cyclic RGDfV (specific antagonist to αvβ3) blocked the adhesion of β3-CHO cells to wt NRG1 (175-222).

FIG. 3. Adhesion of CHO cells that express human β1 (β1-CHO) or β1-3-1 (β1-3-1-CHO) to wt NRG1 (175-222). The β1-3-1 mutation changes the specificity of β1 integrins to that of β3 integrins. Cell adhesion was performed as described herein. β1-3-1-CHO cells adhered to wt NRG1 (175-222) and this binding was blocked by anti-human β1 antibody AIIB2, but not by purified mouse IgG (mIgG). BSA: BSA as a control was coated instead of NRG1 (175-222). The results suggest that NRG1 (175-222) specifically binds to β1-3-1 (as αvβ1-3-1) but not to wt β1 (as αvβ1).

FIG. 4. A model of NRG1-integrin αvβ3 interaction predicted by docking simulation by using AutoDock3. AutoDock 3 is a widely used docking simulation program. The model predicts that the EGF-like domain of NRG1 binds to the RGD-binding site of the integrin αvβ3 headpiece. According to the TGFα-EGFR complex (PDB code 1MOX, EGF and TGFα are homologous), EGFR (homologous to ErbB3 or B4) binds to the side of NRG1 opposite to the predicted integrin-binding site based on the published EGF-EGFR complex structure (not shown). The Lys residues at positions 181, 185, and 187 of NRG1 are located within the binding interface and are well conserved. These residues were chosen for mutagenesis studies.

FIG. 5. The 3KE (175-222) mutant is defective in binding to αvβ3-K562 cells. Adhesion assays was performed using αvβ3-K562 (left) or mock transfected cells (right) as described herein. The data is shown as means+/−SEM of triplicate experiments.

FIG. 6. The 3KE (175-222) mutant is defective in binding to β1-3-1 integrins. Adhesion assays was performed using β1-3-1-CHO cells as described herein. Data are shown as means+/−SEM of triplicate experiments. The results indicate that the 3KE (175-222) mutant fails to bind to αvβ3.

FIG. 7. The wt EGF domain of NRG1 (NRG (175-222)) bound to αvβ3, while the 2KE (175-222) mutant did not. Wt GST-NRG1 (175-222) and the 2KE mutant of GST-NRG1 (175-222) were immobilized to wells of 96-well titer plates. The concentrations of the coating solution are shown. Soluble recombinant integrin αvβ3 (5 μg/ml) was added to the wells in the presence of 1 mM Mn²⁺ and incubated for 2 h at room temperature. After washing, the wells bound αvβ3 was determined by using anti-β3 antibody and HRP-conjugated anti-mouse IgG. The data are shown as means+/−SD of triplicate experiments. Similar results were obtained with the 3KE (175-222) mutant.

FIG. 8. Wt NRG1 (175-222) induced proliferation, but the mutant NRG1 (175-222) did not. The mutant NRG1 (175-222) suppressed proliferation induced by wt NRG1 (175-222) (dominant-negative effect). Human M21 melanoma cells were serum-starved overnight and cultured 48 h with wt or mutant GST-NRG1 (175-222). Cell proliferation was measured by using MTS assays. Data are shown as means+/−SE (n=3). Note that wt GST-NRG1 (175-222) induced cell proliferation while the 3KE (175-222) mutant did not. This indicates that the mutant is defective in inducing mitogenesis. For 3KE+WT, 10 ng/ml wt NRG1 (175-222) and mutant NRG1 (175-222) were added at the indicated concentrations. The data indicate that the 3KE (175-222) mutant blocked the function of wt NRG1 (175-222) (a dominant negative effect). P<0.0001 between wt and 3KE, p=0.0001 between wt and 3KE+wt by 2-way ANOVA. There is no statistical significance between 3KE and 3KE+wt (p=0.58).

FIG. 9. Effect of the 3KE (175-222) mutation on ErbB3 phosphorylation, ERK1/2 activation, and cyclin D1 levels in M21 human melanoma cells. Cells were cultured in DMEM+1% FBS medium for 24 h, and then treated with 3KE or WT NRG1 (175-222) (10 ng/ml, if concentrations were not indicated) for 15 min (if time is not specified). Cell lysates were analyzed by Western blotting. Wt NRG1 (175-222) induced ErbB3 phosphorylation, ERK1/2 activation, while the 3KE (175-222) mutant did not induced ErbB3 phosphorylation, and suppressed ERK1/2 activation, and the levels of cyclin D1.

FIG. 10. Suppression of cell proliferation by 3KE (175-222). B-16 F10 Cells were starved for 5 days in DMEM+1% FBS medium, and then added 3KE (175-222) and WT (175-222) NRG1 at indicated concentrations for 24 hours by MTS assays. P=0.0354 (between wt and 3KE+wt) and P<0.0001 (between wt and 3KE) by 2-way ANOVA.

FIG. 11. 3KE (175-222) suppressed AKT activation in B16F10 melanoma cells. Cells were serum starved and stimulated with 10 ng/ml wt or mutant NRG1 (175-222). Cell lysates were analyzed by western blotting. The results indicate that 3KE (175-222) suppressed AKT activation and D1 cyclin levels.

FIG. 12. Effect of wt and 3KE NRG1 (175-222) on the proliferation of MCF-7 cells. Human MCF-7 breast cancer cells were serum-starved overnight and cultured 48 h with wt or mutant GST-NRG1 (175-222). Cell proliferation was measured by using MTS assays. Note that wt GST-NRG1 (175-222) induced cell proliferation while the 3KE (175-222) mutant did not. This indicates that the mutant is defective in inducing mitogenesis in breast cancer, as in melanoma cells. P=0.0018 by 2-way ANOVA.

FIG. 13. Wt NRG1 (175-222) enhanced Tyr-phosphorylation of the integrin β3 cytoplasmic domain, while 3KE (175-222) suppressed it in M21 cells. Serum-starved M21 cells were stimulated with 10 ng/ml wt or 3KE NRG1 (175-222). Tyr-phosphorylation of β3 was determined by Western blotting of cell lysates. The results indicate that wt NRG1 (175-222) markedly enhanced levels of Tyr-phosphorylation, but 3KE (175-222) suppressed it.

FIG. 14. Wt NRG1 (175-222) suppressed ERK1/2 activation and the 3KE (175-222) mutant enhanced ERK1/2 activation in ErbB4-CHO cells. ErbB4 was stably expressed in CHO cells (designated ErbB4-CHO cells). ErbB4 was detected in a cell lysate of ErbB4-CHO cells by western blotting (top). Serum-starved CHO or ErbB4-CHO cells were stimulated with wt NRG1 (175-222) or the 3KE (175-222) mutant and ERK1/2 activation was measured by Western blotting. The results show that wt NRG1 (175-222) enhanced, and 3KE (175-222) suppressed, ERK1/2 activation in CHO cells (middle). In contrast, wt NRG (175-222) suppressed, and 3KE (175-222) enhanced ERK1/2 activation in ErbB4-CHO cells (bottom).

FIG. 15. 3KE (175-222) mutant suppressed tumorigenesis in a xenograft experiment with highly metastatic Met-1 tumor. Met-1 tumor (a highly metastatic mouse breast cancer) (4×4×4 mm) was transplanted subcutaneously into nude mice. The 3KE (175-222) mutant or vehicle was intraperitoneally injected (100 ng/ml/day/mouse 5 days a week) to mice, starting day 0. Data are shown as means+/−SEM. P<0.0001 by 2-way ANOVA (n=2 for control and n=3 for 3KE).

FIG. 16. Inhibitory effect of daily intraperitoneal injection of 3KE (175-222) on the outgrowth of precancer (MIN-O) in FVB mice. MIN-O was transplanted into fat pads of FVB mice (two per mice) and 3KE (175-222) (200 ng/mouse/day) was injected 5 days a week for 4 weeks starting day 0. After 28 days, mice were sacrificed and the dimensions of tumors were measured. Since MIN-O grows as a thin layer, the two-dimensional size was calculated and the thickness was ignored. P=0.0355 by ANOVA between control and 3KE (175-222).

FIG. 17. CHO cells that express human α6β4 adhered to wt NRG1 (175-222) but not to the 3KE (175-222) mutant. Wells of 96-well microtiter plate were coated with wt or 3KE mutant NRG1 (175-222) and incubated with α6β4-CHO or β1-CHO cells in DMEM for 1 h at 37° C. as described herein. Bound cells were quantified. The results indicate that NRG1 (175-222) is a ligand for α6β4 and 3KE (175-222) is defective in binding to α6β4.

FIG. 18. 3KE (175-222) suppressed levels of ERK1/2 activation in HaCAT cells. HaCAT keratinocytes were serum-starved and incubated with wt NRG1 (175-222) or 3KE (175-222). The levels of Erk1/2 activation were determined by Western blotting. The results indicate that 3KE (175-222) suppressed the Erk1/2 activation, while the background Erk1/2 level was high under the conditions used.

FIG. 19. Docking simulation of αvβ3-NRG1 interaction. a) A model of NRG1α-integrin αvβ3 interaction predicted by docking simulation by using AutoDock3. The headpiece of integrin αvβ3 (PDB code 1LG5) was used as a target. The model predicts that the EGF-like domain of NRG1α binds to the RGD-binding site of the integrin αvβ3 headpiece. b) The Lys residues at positions 181, 185, and 187 of NRG1α are located at the interface between NRG1α and αvβ3, and selected for mutagenesis studies. c) Superposition of TGFα and NRG1. d) The Lys residues at position 181, 185 and 187 of NRG1 are not located in the binding site for EGFR. In the present invention, TGFα in the TGFα-EGFR complex (PDB code 1MOX) was replaced by NRG1 (PDB code 1HAF), by superposing. ErbB3 or ErbB4 is homologous to EGFR.

FIG. 20. The 3KE mutant of NRG1α (175-241) binds to ErbB3. a). Binding of the 3KE mutant of NRG1α (175-241) to recombinant ErbB3. Recombinant soluble ErbB3 Fc fusion protein (R&D system) was coated onto wells of 96-well microtiter plate (1 μg/ml). NRG1α (175-241) WT or 3KE (175-241) mutant was added to the wells and incubated for 1 h at room temperature. GST was used as a control. After washing the wells, bound GST NRG1α (175-241) was determined by using anti-GST antibody HRP conjugate. The results suggest that the 3KE mutant of NRG1α (175-241) binds to ErbB3 at levels nearly comparable to WT NRG1α (175-241). b). Competitive binding assay. Recombinant soluble ErbB3 Fc fusion protein was coated onto well of 96-well microtiter plate (1 μg/ml). Binding of biotin labeled NRG1α (175-241) WT (20 nM) in the presence of increasing concentrations of NRG1α (175-241) WT, 3KE (175-241) or GST. After washing the wells, bound biotin labeled NRG1α (175-241) WT was determined by using streptavidin HRP conjugate. The results suggest that the 3KE mutant of NRG1α (175-241) binds to ErbB3 at levels comparable to WT NRG1α (175-241).

FIG. 21. WT NRG1α (175-241) induced co-precipitation of integrin αvβ3 and ErbB3, while 3KE (175-241) is defective in this function. a) MCF-7 cells were serum-starved overnight and stimulated with WT or 3KE NRG1α (175-241) at indicated time points. In the present invention, 1 mg protein of cell lysate was used for immuno-precipitation of the αvβ3-ErbB3 complex with anti-ErbB3 antibody Immunoprecipitated materials were analyzed by Western blotting using antibodies specific to ErbB3, phosphorylated ErbB3, or integrin β3. The levels of ErbB3 phosphorylation were less with the 3KE mutant (175-241). Integrin αvβ3 was co-immuno-precipitated with the ErbB3 in 5-30 min upon stimulation with WT NRG1α (175-241), while the 3KE (175-241) mutant was defective in this function. b) Levels of β3 coprecipitation. In the present invention, the levels of β3 co-precipitation from digital images was quantified using ImageJ. The data are normalized with time 0 as 1 from two independent experiments. The data demonstrate that the WT NRG1α (175-241) enhanced the association of αvβ3 and ErbB3, but 3KE (175-241) did not, suggesting that this process is dependent on the ability of NRG1α (175-241) to bind to αvβ3.

FIG. 22. Effect of the 3KE mutation on NRG1 signaling. The 3KE mutant of NRG1α (175-241) is defective in inducing ErbB3 phosphorylation, AKT activation and ERK1/2 activation. MCF-7 cells were serum-starved overnight and stimulated with 2.5 nM WT and the 3KE mutant of NRG1α (175-241) at indicated time points. Cell lysates were analyzed by western blotting. WT NRG1α (175-241) markedly induced phosphorylation of ErbB3, AKT, and Erk1/2 (a), while the 3KE (175-241) mutant only weakly induced phosphorylation of ErbB3 and Erk1/2 (b). c-e) Levels of phosphorylation was determined from the digital images (a and b) using Image J and normalized using total protein contents. Data are shown with signals at time 0 as 1.

FIG. 23. Effect of the 3KE (175-241) mutation on in vivo tumorigenesis. Top: Growth curve. Injection of 3KE suppressed tumor growth. (P<0.0001 by two way ANOVA.) Bottom: Tumor size at day 18. 3KE suppressed tumor growth (Statistical analysis was performed by t-test.)

### DEFINITIONS

The term “inhibiting” or “inhibition,” as used herein, refers to any detectable negative effect on a target biological process, such as the binding between NRG1 and integrin αvβ3, α6β4, αβ1 or α9β1, or on its downstream processes including ErbB3 phosphorylation, phosphorylation of integrin cytoplasmic domain, ERK1/2 activation, cyclin D1 expression, and AKT activation, as well as cell proliferation, tumorigenicity, and metastatic potential. Typically, an inhibition is reflected in a decrease of at least 10%, 20%, 30%, 40%, or 50% in NRG1-integrin binding, or any one of the downstream parameters mentioned above, when compared to a control.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N.Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example, a core amino acid sequence responsible for NRG-integrin binding has at least 80% identity, preferably 85%, 90%, 91%, 92%, 93, 94%, 95%, 96%, 97%, 98%, 99%, or 100% identity, to a reference sequence, e.g., SEQ ID NO:1), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “effective amount,” as used herein, refers to an amount that produces therapeutic effects for which a substance is administered. The effects include the prevention, correction, or inhibition of progression of the symptoms of a disease/condition and related complications to any detectable extent. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

As used herein, a “polypeptide comprising the NRG-1 integrin binding region” refers to a polypeptide containing a core amino acid sequence that generally corresponds to the amino acid sequence of SEQ ID NO:1. This core amino acid sequence may contain some variations such as amino acid deletion, addition, or substitution, but should maintain a substantial level sequence homology (e.g., at least 80%, 85%, 90%, 95%, or higher sequence homology) to SEQ ID NO:1 and is capable of binding integrin αvβ3, α6β4, α6β1 or α9β1. In addition to this core sequence that is responsible for the polypeptide's ability to bind to integrin, one or more amino acid sequences of a homologous origin (e.g., additional sequence from the same protein, NRG-1) or a heterologous origin (e.g., sequence from another unrelated protein) can be included in the polypeptide. Some examples of the “polypeptide comprising the NRG-1 integrin binding site” include SEQ ID NO:1, SEQ ID NO:2, and the full length wild type NRG-1. Optionally, an affinity or epitope tag (such as a GST tag) can be included in the polypeptide to facilitate purification, isolation, or immobilization of the polypeptide.

An “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′_{2,} a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology.

Further modification of antibodies by recombinant technologies is also well known in the art. For instance, chimeric antibodies combine the antigen binding regions (variable regions) of an antibody from one animal with the constant regions of an antibody from another animal. Generally, the antigen binding regions are derived from a non-human animal, while the constant regions are drawn from human antibodies. The presence of the human constant regions reduces the likelihood that the antibody will be rejected as foreign by a human recipient. On the other hand, “humanized” antibodies combine an even smaller portion of the non-human antibody with human components. Generally, a humanized antibody comprises the hypervariable regions, or complementarity determining regions (CDR), of a non-human antibody grafted onto the appropriate framework regions of a human antibody. Antigen binding sites may be wild type or modified by one or more amino acid substitutions, e.g., modified to resemble human immunoglobulin more closely. Both chimeric and humanized antibodies are made using recombinant techniques, which are well-known in the art (see, e.g., Jones et al. (1986) Nature 321:522-525).

Thus, the term “antibody,” as used herein, also includes antibody fragments either produced by the modification of whole antibodies or antibodies synthesized de novo using recombinant DNA methodologies (e.g., single chain Fv, a chimeric or humanized antibody).

### DETAILED DESCRIPTION OF THE INVENTION

### I. Introduction

The neuregulins (NRGs) are a family of signaling proteins that mediates interaction between cells in the heart, breast, and central and peripheral nervous systems. They contain an epidermal growth factor (EGF)-like motif that binds to and activates receptor tyrosine kinases in the EGF receptor (ErbBs) family. NRG1 is involved in cell fate, cell migration, and cell differentiation in the developing and adult nervous system. NRG1 regulates the functional expression of ligand and voltage gated channels in neurons; the formation, maturation and maintenance of the developing neuromuscular junction; and the proliferation, survival and differentiation of glia. NRG1 also acts as a chemoattractant and contact-dependent guide for interneurons in the developing CNS. The EGF receptor (EGFR, ErbBs) family consists of ErbB1, ErbB2, ErbB3 and ErbB4, which differ in their ability to bind ligand or elicit a signal. ErbB2 has no direct ligand. In comparison, ErbB3 can bind NRG1, but it is lacking intracellular kinase activity. The ErbB4 receptor has the ability to bind NRG1 and also contains a highly active tyrosine kinase domain. Once NRG1 is bound, it stimulates homologous and heterologous dimerization of EGF receptor family members, leading to the phosphorylation of tyrosine residues. ErbB3 can dimerize with ErbB2 and ErbB4, but it is the dimerization of ErbB2 with ErbB4 that can form the highest affinity-binding site and greatly enhance the level of tyrosine phosphorylation. In vivo, functional NRG1 receptors are heterodimers composed of ErbB2 with either an ErbB3, or ErbB4 molecule.

Evidence implicates the aberrant activation of ErbB receptors in the progression of various human tumors, notably breast and ovarian cancers. Overexpression of EGF receptor, ErbB2, and ErbB3 has been observed in numerous solid tumors types, and correlates with a high degree of receptor activation. Amplification of the erbB2 gene is observed in 25-30% of breast cancer patients, and overexpression of the product correlates with earlier relapse and poor prognosis. The observed efficacy of the FDA-approved drug trastuzumab (Genentech's Herceptin), a humanized antibody directed to the ErbB2 (HER2/neu) protein, toward ErbB2-positive breast tumors validates this receptor as a therapeutic target.

Since the aberrant activation of ErbB2 protein tyrosine kinase activity is thought to contribute to tumor progression by engaging specific cellular signaling pathways that promote progression, much emphasis has been placed on understanding the biochemical mechanisms by which ErbB2 and its relatives are activated. The members of the ErbB receptor family undergo a network of homo- and heterodimerization events as part of their activation mechanism. Particularly noteworthy is a strong propensity of ErbB2 to heterodimerize with and activate ErbB3, especially when the two receptors are overexpressed. Studies have established a strong link between the coordinate overexpression and activation of ErbB2 and ErbB3 in breast tumor cell lines and in patient samples. Moreover, in tumors from transgenic mice generated by expressing an active allele of ErbB2, ErbB3 overexpression and activation is also observed. On the basis of such expression studies it has been suggested that the ErbB3 receptor may also be used as a marker for patient prognosis, and that ErbB3 may contribute to the progression of breast tumor cells from non-invasive to invasive. In vitro, ErbB2 and ErbB3 synergize in promoting the growth and transformation of cultured fibroblasts, and numerous studies demonstrate that the two receptors synergize in mediating increased invasiveness induced by NRG1 in breast tumor cell lines. Taken together, these observations suggest that there may be an advantage for both receptors to be present and activated in tumor cells to promote breast tumor growth and progression.

In the present invention, novel ways to block NRG/ErbB signaling in breast cancer were explored. Integrins have been shown to crosstalk with receptor tyrosine kinase (RTK) in growth factor signaling. Integrins are a family of cell adhesion receptors that recognize extracellular matrix ligands and cell surface ligands. Integrins are transmembrane α-β heterodimers, and at least 18α and 8β subunits are known. Integrins are involved in signal transduction upon ligand binding, and their functions are in turn regulated by signals from within the cell. It has been reported that there is a positive correlation between αvβ3 integrin levels and overexpression of NRG associated with breast cancer tumor progression and metastasis. It has been proposed that NRG1 may play a key role in the regulation of αvβ3 integrin expression and in its signaling functions. However, the specifics of the role of integrins in NRG1/ErbB signaling are unclear.

In the present invention, it was discovered that the EGF-like domain of NRG1 directly binds to integrin αvβ3 (and perhaps other integrins). A GST fusion protein of the NRG1 EGF-like domain was generated. The EGF-like domain of the NRGs is known to be sufficient to specifically activate ErbB receptors and induce cellular responses in culture. In the present invention, integrin-binding-defective mutants of NRG1 (designated 3KE and 2KE mutants) were generated, and it was found, in the present invention, that the 3KE mutant did not induce cell proliferation, and suppressed cell proliferation induced by wt NRG1 (dominant-negative effect by definition). The position of the 3KE mutation is distinct from that of ErbB-binding site. These results indicate that the 3KE mutant has an immediate use as an anti-tumor therapeutic.

### II. Production of NRG-Related Polypeptides

A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a neuregulin gene, a polynucleotide encoding a polypeptide comprising the integrin-binding sequence of NRG1, and synthetic oligonucleotides can be verified after cloning or subcloning using, e.g., the chain termination method for sequencing double-stranded templates of Wallace et al., Gene 16: 21-26 (1981).

B. Coding Sequence for a NRG-Related Polypeptide

Polynucleotide sequences encoding human neuregulin, e.g., GenBank Accession No. M94165, have been determined and may be obtained from a commercial supplier.

The rapid progress in the studies of human genome has made possible a cloning approach where a human DNA sequence database can be searched for any gene segment that has a certain percentage of sequence homology to a known nucleotide sequence, such as one encoding a previously identified human neuregulin. Any DNA sequence so identified can be subsequently obtained by chemical synthesis and/or a polymerase chain reaction (PCR) technique such as overlap extension method. For a short sequence, completely de novo synthesis may be sufficient; whereas further isolation of full length coding sequence from a human cDNA or genomic library using a synthetic probe may be necessary to obtain a larger gene.

Alternatively, a nucleic acid sequence encoding a human neuregulin can be isolated from a human cDNA or genomic DNA library using standard cloning techniques such as polymerase chain reaction (PCR), where homology-based primers can often be derived from a known nucleic acid sequence encoding a neuregulin. Most commonly used techniques for this purpose are described in standard texts, e.g., Sambrook and Russell, supra.

cDNA libraries suitable for obtaining a coding sequence for a human neuregulin may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full length polynucleotide sequence encoding the neuregulin from the cDNA library. A general description of appropriate procedures can be found in Sambrook and Russell, supra.

A similar procedure can be followed to obtain a full-length sequence encoding a human neuregulin, e.g., any one of the GenBank Accession Nos. mentioned above, from a human genomic library. Human genomic libraries are commercially available or can be constructed according to various art-recognized methods. In general, to construct a genomic library, the DNA is first extracted from a tissue where a neuregulin is likely found. The DNA is then either mechanically sheared or enzymatically digested to yield fragments of about 12-20 kb in length. The fragments are subsequently separated by gradient centrifugation from polynucleotide fragments of undesired sizes and are inserted in bacteriophage λ vectors. These vectors and phages are packaged in vitro. Recombinant phages are analyzed by plaque hybridization as described in Benton and Davis, Science, 196: 180-182 (1977). Colony hybridization is carried out as described by Grunstein et al., Proc. Natl. Acad. Sci. USA, 72: 3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, the full-length nucleic acid encoding a neuregulin is obtained.

Upon acquiring a nucleic acid sequence encoding a neuregulin, the coding sequence can be further modified by a number of well known techniques such as restriction endonuclease digestion, PCR, and PCR-related methods to generate coding sequences for neuregulin-related polypeptides, including neuregulin mutants (especially the dominant negative type) and polypeptides comprising an integrin-binding sequence derived from a neuregulin. The polynucleotide sequence encoding a desired neuregulin-related polypeptide can then be subcloned into a vector, for instance, an expression vector, so that a recombinant polypeptide can be produced from the resulting construct. Further modifications to the coding sequence, e.g., nucleotide substitutions, may be subsequently made to alter the characteristics of the polypeptide.

A variety of mutation-generating protocols are established and described in the art, and can be readily used to modify a polynucleotide sequence encoding a NRG-related polypeptide. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other possible methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

C. Modification of Nucleic Acids for Preferred Codon Usage in a Host Organism

The polynucleotide sequence encoding a neuregulin-related polypeptide can be further altered to coincide with the preferred codon usage of a particular host. For example, the preferred codon usage of one strain of bacterial cells can be used to derive a polynucleotide that encodes a recombinant polypeptide of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of the neuregulin-related polypeptides.

D. Chemical Synthesis of NRG-Related Polypeptides

The amino acid sequence of integrin-bind site derived from human neuregulin 1 (NRG1) has been established as SEQ ID NO:1, and can be as short as residues 181-187 of SEQ ID NO:4. A polypeptide comprising this NRG1-integrin binding sequence thus can also be chemically synthesized using conventional peptide synthesis or other protocols well known in the art.

Polypeptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an a-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

### III. Expression and Purification of NRG-Related Polypeptides

Following verification of the coding sequence, a NRG-related polypeptide of the present invention can be produced using routine techniques in the field of recombinant genetics, relying on the polynucleotide sequences encoding the polypeptide disclosed herein.

A. Expression Systems

To obtain high level expression of a nucleic acid encoding a NRG-related polypeptide of the present invention, one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the NRG-related polypeptide in host cells. A typical expression cassette thus contains a promoter operably linked to the nucleic acid sequence encoding the NRG-related polypeptide and signals required for efficient polyadenylation of the transcript, ribosome binding sites, and translation termination. The nucleic acid sequence encoding the NRG-related polypeptide is typically linked to a cleavable signal peptide sequence to promote secretion of the polypeptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the NRG-related polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant protein (e.g., a NRG-related polypeptide of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

A person skilled in the art will recognize that various conservative substitutions can be made to any wild-type or mutant neuregulin or a polypeptide comprising an integrin-binding sequence of neuregulin while still retaining the biological activity of the polypeptide, e.g., the ability to bind integrin and/or promote or inhibit ErbB signaling. Moreover, modifications of a polynucleotide coding sequence may also be made to accommodate preferred codon usage in a particular expression host without altering the resulting amino acid sequence.

B. Transfection Methods

Standard transfection methods are used to produce bacterial, mammalian, yeast, insect, or plant cell lines that express large quantities of a NRG-related polypeptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the NRG-related polypeptide.

C. Purification of Recombinantly Produced NRG-Related Polypeptides

Once the expression of a recombinant NRG-related polypeptide in transfected host cells is confirmed, e.g., via an immunoassay such as Western blotting assay, the host cells are then cultured in an appropriate scale for the purpose of purifying the recombinant polypeptide.

1. Purification of Recombinantly Produced Polypeptides from Bacteria

When the NRG-related polypeptides of the present invention are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Additional methods of lysing bacteria are described in Ausubel et al. and Sambrook and Russell, both supra, and will be apparent to those of skill in the art.

The cell suspension is generally centriftiged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides, e.g., a NRG-related polypeptide, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

2. Standard Protein Separation Techniques for Purification

When a recombinant polypeptide of the present invention, e.g., a neuregulin mutant or a polypeptide comprising a NRG-integrin binding sequence, is expressed in host cells in a soluble form, its purification can follow the standard protein purification procedure described below. This standard purification procedure is also suitable for purifying NRG-related polypeptides obtained from chemical synthesis.

i. Solubility Fractionation

Often as an initial step, and if the protein mixture is complex, an initial salt fractionation can separate many of the unwanted host cell proteins (or proteins derived from the cell culture media) from the recombinant protein of interest, e.g., a NRG-related polypeptide of the present invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

ii. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a NRG-related polypeptide. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

iii. Column Chromatography

The proteins of interest (such as a NRG-related polypeptide of the present invention) can also be separated from other proteins on the basis of their size, net surface charge, hydrophobicity, or affinity for ligands. In addition, antibodies raised against a segment of NRG such as the integrin-binding site can be conjugated to column matrices and the NRG-related polypeptide immunopurified. All of these methods are well known in the art.

It will be apparent to one of skill that chromatographic techniques can be performed at any scale and using equipment from many different manufacturers (e.g., Pharmacia Biotech).

### IV. Identification of Inhibitors for NRG-Integrin Binding

A. Neuregulin-Integrin Binding Assays

An in vitro assay can be used to detect neuregulin-integrin binding and to identify compounds that are capable of inhibiting neuregulin-integrin binding. In general, such an assay can be performed in the presence of a neuregulin, such as human neuregulin 1 (e.g., neuregulin 1α (NRG1α) or neuregulin 1β (NRG1β)), and an integrin, such as αvβ3, α6β4, α6β1 or α9β1, that are known to bind each other, under conditions permitting such binding. For convenience, one of the binding partners may be immobilized onto a solid support and/or labeled with a detectable moiety. A third molecule, such as an antibody (which may include a detectable label) to one of the binding partners, can also be used to facilitate detection.

In some cases, the binding assays can be performed in a cell-free environment; whereas in other cases, the binding assays can be performed on cell surface, frequently using cells recombinantly or endogenously expressing an appropriate integrin molecule. More details and some examples of such binding assays can be found in the Examples section of this application.

To screen for compounds capable of inhibiting neuregulin-integrin binding, the above-described assays are performed both in the presence and absence of a test compound, the level of neuregulin-integrin binding is then compared. If neuregulin-integrin binding is suppressed at the presence of the test compound at a level of at least 10%, more preferably at least 20%, 30%, 40%, or 50%, or even higher, the test compound is then deemed an inhibitor of neuregulin-integrin binding and may be subject to further testing to confirm its ability to inhibit ErbB signaling.

The binding assay is also useful for confirming that a polypeptide comprising an integrin-binding sequence derived from a neuregulin can indeed specifically bind integrin. For instance, a polypeptide comprising SEQ ID NO:1 (or residues 181-187 of SEQ ID NO:4) but not the full length NRG1 sequence may be recombinantly expressed, purified, and placed in a binding assay with integrin αvβ3, α6β4, α6β1 or α9β1, substituting a full length wild type NRG1 protein, which is used in a control assay to provide a comparison basis. If deemed to have sufficient integrin-binding ability, a polypeptide comprising an NRG1-integrin binding sequence can then be used, in place of a wild-type full length NRG1 protein, in a binding assay for identifying inhibitors of NRG1-integrin binding. Similarly, a polypeptide comprising a core sequence with a high level of homology (e.g., 90%, 95% or higher) to SEQ ID NO:1 (or residues 181-187 of SEQ ID NO:4) can be tested and, if appropriate, can be used, in place of a wild-type full length NRG1 protein, in a binding assay for identifying inhibitors of NRG1-integrin binding.

Inhibitors of NRG1-integrin binding can have diverse chemical and structural features. For instance, an inhibitor can be a non-functional NRG1 mutant that retaining integrin-binding ability, an antibody to either NRG1 or integrin that interferes with NRG1-integrin binding, or any small molecule or macromolecule that simply hinders the interaction between NRG1 and integrin. Essentially any chemical compound can be tested as a potential inhibitor of NRG1-integrin binding. Most preferred are generally compounds that can be dissolved in aqueous or organic (especially DMSO-based) solutions Inhibitors can be identified by screening a combinatorial library containing a large number of potentially effective compounds. Such combinatorial chemical libraries can be screened in one or more assays, as described herein, to identify those library members (particular chemical species or subclasses) that display a desired characteristic activity. The compounds thus identified can serve as conventional “lead compounds” or can themselves be used as potential or actual therapeutics.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)) and carbohydrate libraries (see, e.g., Liang et al., Science, 274:1520-1522 (1996) and U.S. Pat. No. 5,593,853). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514).

B. ErbB Signaling Assays

The inhibitors of neuregulin-integrin binding are useful for their ability to inhibit ErbB signaling, especially as anti-cancer therapeutics for cancer patients overexpressing one or more ErbB members. Assays for confirming such inhibitory effect of an inhibitor can be performed in vitro or in vivo. An in vitro assay typically involves exposure of cultured cells to an inhibitor and monitoring of subsequent biological and biochemical changes in the cells. For example, following exposure to 0.1-20 μg/ml an inhibitor for 0.5-48 hours, suitable cells (such as those expressing integrin αvβ3, α6β4, α6β1 or α9β1 and responsive to heregulin) are examined for their proliferation/survival status using methods such as direct cell number counting, BrdU or H³-thymidine incorporation, tetrazolium salt 3,[4,5-dimethylthiazol-2-yl]-2,5-diphenyltetrazolium bromide (MTT) cell proliferation assay, 3-(4,5-dimethylthiazol-2-yl)-5-(3-carboxymethoxyphenyl)-2-(4-sulfophenyl)-2H-tetrazolium (MTS) cell proliferation assay, chicken embryo allantoic membrane (CAM) assay, TUNNEL assay, annexin V binding assay, etc. Further downstream changes due to ErbB signaling, e.g., phosphorylation of ErbB3 or integrin cytoplasmic domain, ERK1/2 activation, cyclin D1 expression, and AKT activation can also be monitored to provide an indication of suppressed ErbB signaling. In addition, tumorigenicity of cancer cells is useful parameters for monitoring and can be tested by methods such as colony formation assays or soft agar assays. Detailed description of some exemplary assays can be found in the Examples section of this disclosure. An inhibitory effect is detected when a decrease in ErbB signaling, as indicated by any one aforementioned parameter, of at least 10%, 20%, 30%, 40%, 50%, 60%, 70%, 80%, 90% or more is observed.

The effects of a NRG1-integrin binding inhibitor of the present invention can also be demonstrated in in vivo assays. For example, an inhibitor of NRG1-integrin can be injected into animals that have a compromised immune system (e.g., nude mice, SCID mice, or NOD/SCID mice) and therefore permit xenograft tumors. Injection methods can be subcutaneous, intramuscular, intravenous, intraperitoneal, or intratumoral in nature. Tumors development is subsequently monitored by various means, such as measuring tumor volume and scoring secondary lesions due to metastases, in comparison with a control group of animals with similar tumors but not given the inhibitors. The Examples section of this disclosure provides detailed description of some exemplary in vivo assays. An inhibitory effect is detected when a negative effect on tumor growth or metastasis is established in the test group. Preferably, the negative effect is at least a 10% decrease; more preferably, the decrease is at least 20%, 30%, 40%, 50%, 60%, 70%, 80%, or 90%.

### V. Pharmaceutical Compositions and Administration

The present invention also provides pharmaceutical compositions or physiological compositions comprising an effective amount of a compound that inhibits neuregulin-integrin binding, such as a dominant negative NRG1 mutant 3KE or its encoding nucleic acid, anti-b3 antibody 7E3 (Coller, B. S. (1985) J. Clin. Invest. 76, 101-108) or cyclic RGDfV (Aumailley et al., (1991) FEBS Lett. 291(1), 50-54) inhibiting ErbB signaling in both prophylactic and therapeutic applications. Such pharmaceutical or physiological compositions also include one or more pharmaceutically or physiologically acceptable excipients or carriers. Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The pharmaceutical compositions of the present invention can be administered by various routes, e.g., oral, subcutaneous, transdermal, intramuscular, intravenous, or intraperitoneal. The preferred routes of administering the pharmaceutical compositions are local delivery to an organ or tissue suffering from a condition exacerbated by ErbB overexpression (e.g., intratumor injection to a tumor) at daily doses of about 0.01-5000 mg, preferably 5-500 mg, of a NRG-integrin binding inhibitor for a 70 kg adult human per day. The appropriate dose may be administered in a single daily dose or as divided doses presented at appropriate intervals, for example as two, three, four, or more subdoses per day.

For preparing pharmaceutical compositions containing a NRG-integrin inhibitor, inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., a NRG dominant negative mutant polypeptide. In tablets, the active ingredient (an inhibitor of NRG-integrin binding) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient of an inhibitor of neuregulin-integrin binding. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of a NRG-integrin binding inhibitor with encapsulating material as a carrier providing a capsule in which the inhibitor (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., a dominant negative NRG mutant polypeptide) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a NRG-integrin binding inhibitor) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing neuregulin-integrin binding inhibitors can be administered for prophylactic and/or therapeutic treatments. In therapeutic applications, compositions are administered to a patient already suffering from a condition that may be exacerbated by the overexpression of ErbB family members in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 2,000 mg of the inhibitor per day for a 70 kg patient, with dosages of from about 5 mg to about 500 mg of the inhibitor per day for a 70 kg patient being more commonly used.

In prophylactic applications, pharmaceutical compositions containing neuregulin-integrin binding inhibitors are administered to a patient susceptible to or otherwise at risk of developing a disease or condition in which overexpression of ErbB is undesirable, in an amount sufficient to delay or prevent the onset of the symptoms. Such an amount is defined to be a “prophylactically effective dose.” In this use, the precise amounts of the inhibitor again depend on the patient's state of health and weight, but generally range from about 0.1 mg to about 2,000 mg of the inhibitor for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of a neuregulin-integrin binding sufficient to effectively inhibit ErbB signaling in the patient, either therapeutically or prophylatically.

### VI. Therapeutic Applications Using Nucleic Acids

A variety of diseases can be treated by therapeutic approaches that involve introducing a nucleic acid encoding a polypeptide inhibitor of integrin-neuregulin binding into a cell such that the coding sequence is transcribed and the polypeptide inhibitor is produced in the cell. Diseases amenable to treatment by this approach include a broad spectrum of solid tumors, the survival and growth of which rely on to some extent the continue signaling of ErbB family members. For discussions on the application of gene therapy towards the treatment of genetic as well as acquired diseases, see, Miller Nature 357:455-460 (1992); and Mulligan Science 260:926-932 (1993).

A. Vectors for Gene Delivery

For delivery to a cell or organism, a polynucleotide encoding a polypeptide that inhibits NRG-integrin binding (such as the dominant negative mutant 3KE) can be incorporated into a vector. Examples of vectors used for such purposes include expression plasmids capable of directing the expression of the nucleic acids in the target cell. In other instances, the vector is a viral vector system wherein the polynucleotide is incorporated into a viral genome that is capable of transfecting the target cell. In a preferred embodiment, the polynucleotide encoding a polypeptide inhibitor can be operably linked to expression and control sequences that can direct expression of the polypeptide in the desired target host cells. Thus, one can achieve expression of the polypeptide inhibitor under appropriate conditions in the target cell.

B. Gene Delivery Systems

Viral vector systems useful in the expression of a polypeptide inhibitor of NRG-integrin binding include, for example, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the genes of interest (e.g., one encoding for a polypeptide inhibitor of the present invention) are inserted into such vectors to allow packaging of the gene construct, typically with accompanying viral DNA, followed by infection of a sensitive host cell and expression of the gene of interest.

As used herein, “gene delivery system” refers to any means for the delivery of a nucleic acid of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221, WO 93/14188, and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, molecular conjugates of the instant invention can include microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors may also be useful for introducing the coding sequence of a polypeptide inhibitor of the invention into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A. 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Patent 4,405,712, Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide or polynucleotide of the invention and thus restore the cells to a normal phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

C. Pharmaceutical Formulations

When used for pharmaceutical purposes, the nucleic acid encoding a neuregulin-integrin binding inhibitor polypeptide is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

D. Administration of Formulations

The formulations containing a nucleic acid encoding a polypeptide inhibitor of the binding between neuregulin and integrin can be delivered to any tissue or organ using any delivery method known to the ordinarily skilled artisan. In some embodiments of the invention, the nucleic acids encoding the inhibitor polypeptides are formulated for subcutaneous, intramuscular, intravenous, intraperitoneal, or intratumor injection.

The formulations containing the nucleic acid of the invention are typically administered to a cell. The cell can be provided as part of a tissue, such as an epithelial membrane, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced into cells by such methods as microinjection, calcium phosphate precipitation, liposome fusion, ultrasound, electroporation, or biolistics. In further embodiments, the nucleic acids are taken up directly by the tissue of interest.

In some embodiments of the invention, the nucleic acids of the invention are administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. To practice the present invention, doses ranging from about 10 ng-1 g, 100 ng-100 mg, 1 ng-10 mg, or 30-300 μg DNA per patient are typical. Doses generally range between about 0.01 and about 50 mg per kilogram of body weight, preferably between about 0.1 and about 5 mg/kg of body weight or about 10⁸-10¹⁰ or 10¹² particles per injection. In general, the dose equivalent of a naked nucleic acid from a vector is from about 1 μg-100 μg for a typical 70 kg patient, and doses of vectors which include a retroviral particle are calculated to yield an equivalent amount of nucleic acid encoding a polypeptide that inhibits the binding between integrin and neuregulin (e.g., human NRG1).

### VII. Kits

The invention also provides kits for inhibiting ErbB signaling according to the method of the present invention. The kits typically include a container that contains a pharmaceutical composition having an effective amount of an inhibitor of neuregulin-integrin binding (such as a dominant negative NRG1 mutant 3KE or a polynucleotide sequence encoding the polypeptide) as well as informational material containing instructions on how to dispense the pharmaceutical composition, including description of the type of patients who may be treated (e.g., cancer patients with ErbB overexpression), the schedule (e.g., dose and frequency) and route of administration, and the like.

### EXAMPLES

The following examples are provided by way of illustration only and not by way of limitation. Those of skill in the art will readily recognize a variety of non-critical parameters that could be changed or modified to yield essentially the same or similar results.

Examples 1-11 were carried out using wild type human neuregulin 1 fragments containing residues 175-222 of SEQ ID NO. 4, and mutants thereof. 3KE or 3KE (175-222) in Examples 1-11 has an amino acid sequence of: GTSHLVECAEEEETFCVNGGECFMVKDLSNPSRYLCKCQPGFTGARCT (SEQ ID NO:11). 2KE or 2KE (175-222) in Examples 1-11 has an amino acid sequence of:

(SEQ ID NO: 12)
GTSHLVKCAEEEETFCVNGGECFMVKDLSNPSRYLCKCQPGFTGARCT.

### Example 1

### Direct Binding of Integrin αvβ3 to NRG1 (175-222)

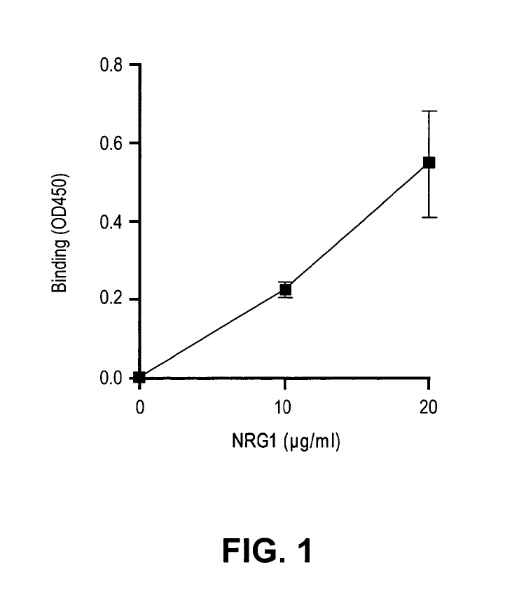

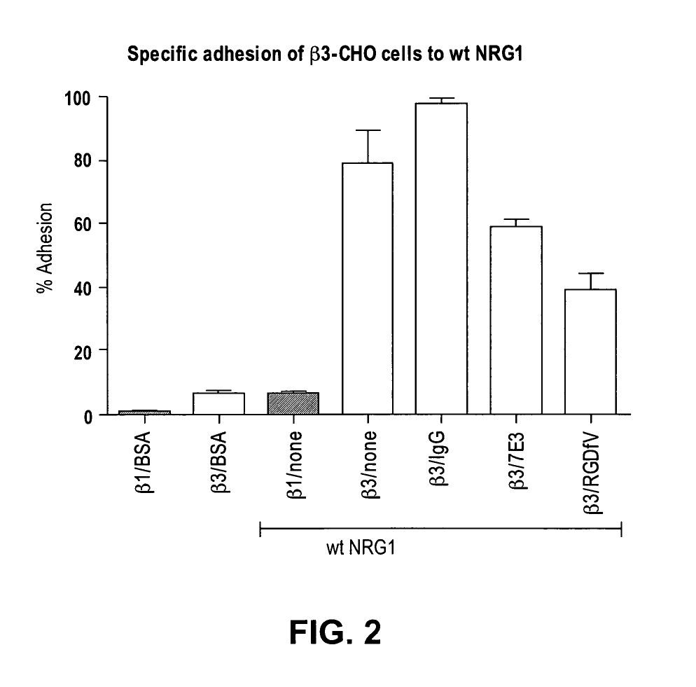

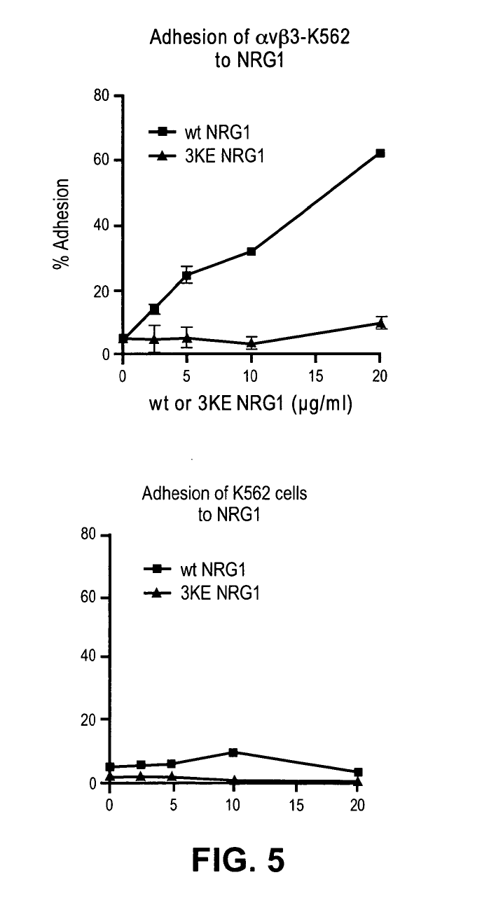

Direct αvβ3-NRG (175-222) interaction: It has been well established that the EGF-like domain of the NRGs is sufficient to specifically activate ErbB receptors and to induce cellular responses in culture. To generate GST-NRG1 EGF-like domain fusion protein, the cDNA fragment of the EGF-like domain of NRG1 was amplified by PCR and subcloned into PGEX-2T vector to generate GST-NRG1 EGF-like domain fusion protein (designated GST-NRG1 (175-222)) in E. coli BL21. The fusion protein was purified in glutathione-affinity chromatography. The affinity resin was extensively washed with 1% Triton X-114 to remove endotoxin before eluting the protein. It was tested, in the present invention, whether soluble recombinant integrin αvβ3 binds to GST-NRG1 (175-222) that is immobilized to plastic wells (ELISA-type assay). Recombinant soluble αvβ3 integrin bound to the 3KE (175-222) mutant (isolated EGF-like domain) in a dose-dependent manner (FIG. 1). It was found, in the present invention, that adhesion of Chinese hamster ovary (CHO) cells that express recombinant β3 (β3-CHO) adhered to NRG1 (175-222), while CHO cells that express recombinant human β1 (β1-CHO) did not (FIG. 2). It was also found, in the present invention, that K562 erythroleukemic cells expressing recombinant αvβ3 (αvβ3-K562) adhered in a dose-dependent manner, but mock-transfected K562 cells did not (see below, FIG. 5). These results indicate that the EGF-like domain of NRG1 (NRG (175-222)) directly interacts with integrin αvβ3. Furthermore, anti-β3 mAb (7E3) and cyclic RGDfV (an-αvβ3-specific antagonist) blocked the adhesion. These results indicate that binding of NRG1 (175-222) to αvβ3 is specific.

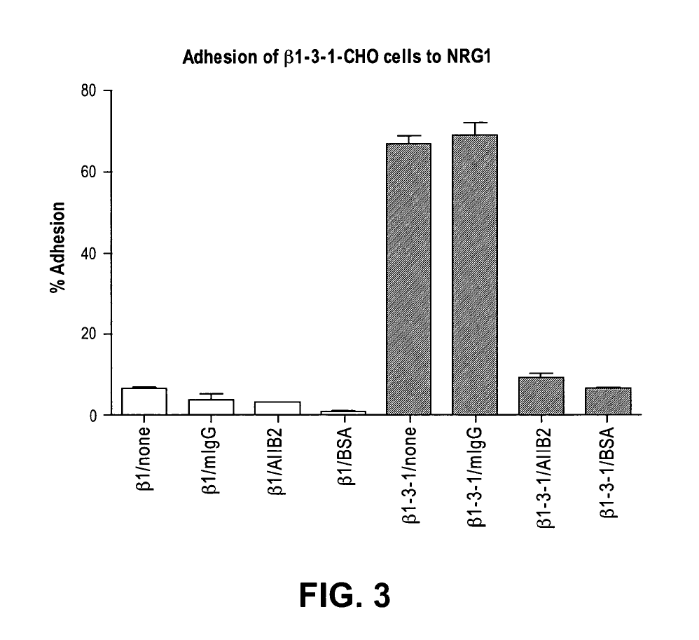

The β1-3-1 mutation: To determine if NRG1 (175-222) binds to αvβ3 like other known αvβ3 ligands (e.g., vitronectin and fibrinogen), a mutant of β1 having a ligand specificity of β3 (Takagi et al. 1997) was used. The β integrin subunit possesses an I-like domain that plays a critical role in ligand binding (Luo et al. 2007). It was shown that when a disulfide-linked five-residue sequence of β1 I-like domain (residues 177-183) of αvβ1 is switched with a corresponding sequence in β3 integrin (designated the β1-3-1 mutant), ligand-binding specificity of the mutated integrin αvβ1-3-1 is altered to that of αvβ3 (Takagi et al. 1997). Hence the loop was designated “the specificity loop.” The β1-3-1 mutant (as αvβ1-3-1) bound to vitronectin and fibrinogen, but wt β1 (as αvβ1) did not (Takagi et al. 1997). It was found that CHO cells expressing β1-3-1 (designated β1-3-1-CHO cells) bound to NRG1 (175-222), but wt β1-CHO did not (FIG. 3). The adhesion of β1-3-1-CHO cells to NRG1 (175-222) was blocked anti-β1 antibody AIIB2 (FIG. 3). The crystal structure of αvβ3 showed that the specificity loop is located in the ligand-binding (RGD-binding) site and undergoes marked conformational changes (1 angstrom shift) upon RGD binding to αvβ3 (Xiong et al. 2002). These findings indicate that NRG1 (175-222) binds to the ligand-binding site of αvβ3, and that the specificity loop plays a role in NRG1 binding to αvβ3.

### Example 2

### Integrin-Binding Defective Mutant of NRG1

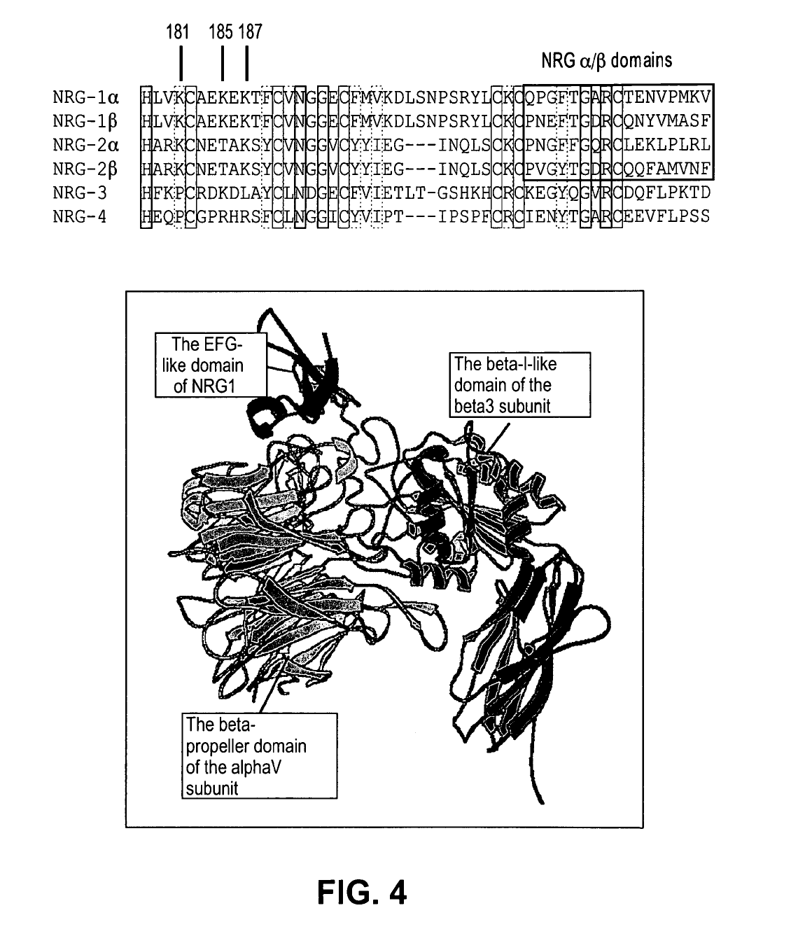

Docking simulation and mutagenesis: To locate the integrin-binding site in NRG1 and to generate integrin-binding-defective mutants of NRG 1, docking simulation and site-directed mutagenesis were used. Docking simulation of the interaction between integrin αvβ3 and the EGF-like domain of NRG1 predicts that NRG binds to integrin αvβ3 with a high affinity (docking energy −23.5 kcal/mol), which is consistent with our binding results. Also the simulation predicted that the integrin-binding site is distinct from the EGFR-binding site of NRG using the TGFα-EGFR complex, PDB code 1MOX, since EGF and TGFα are homologous. The simulation suggests that integrins and ErbB receptors do not block access of NRG1 to each other (FIG. 4). The predicted integrin-binding interface includes Lys residues at positions 181, 185, and 187, which are conserved in NRG1 and NRG2. A NRG mutant that does not bind to integrins was generated by mutating of Lys181/Lys185/Lys187 simultaneously to Glu residues (designated the 3KE (175-222) mutation). The mutated Lys residues are located on the side opposite to the ErbB-binding site, indicating that the integrin-binding-defective mutant may interact with ErbB.

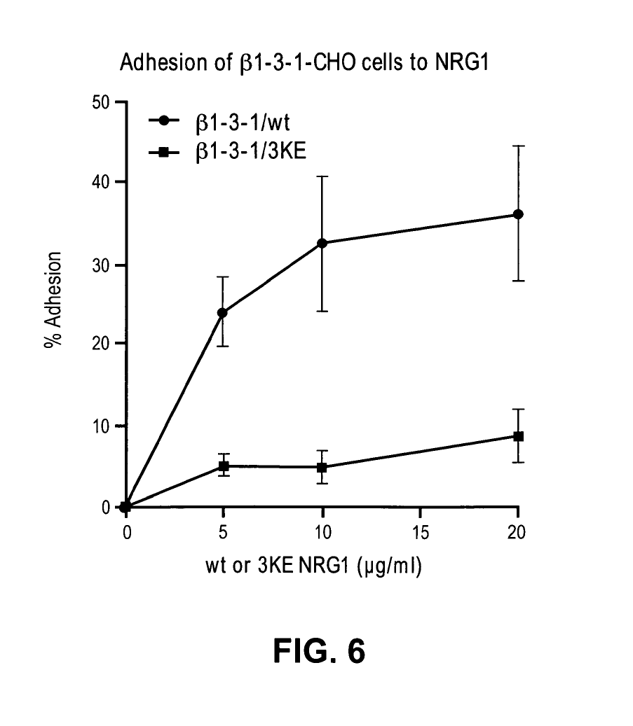

The 3KE (175-222) mutant was tested for its ability to bind to integrin αvβ3 in cell adhesion assays using αvβ3-K562 (FIG. 5) and β1-3-1-CHO cells (FIG. 6). It was found that αvβ3 bound to wt NRG1 (175-222) but did not bind to the 3KE (175-222), indicating that the 3KE (175-222) mutant was defective in binding to αvβ3.

These results suggest that 1) NRG1 (175-222) bound to αvβ3 as predicted by docking simulation, 2) the Lys residues in the predicted integrin-binding site are critical for integrin binding, and 3) the 3KE (175-222) mutation is defective in integrin binding and therefore useful for studying the role of integrins in NRG1/ErbB signaling.

### Example 3

### Integrin-Binding-Defective Mutants of NRG1 (175-222)

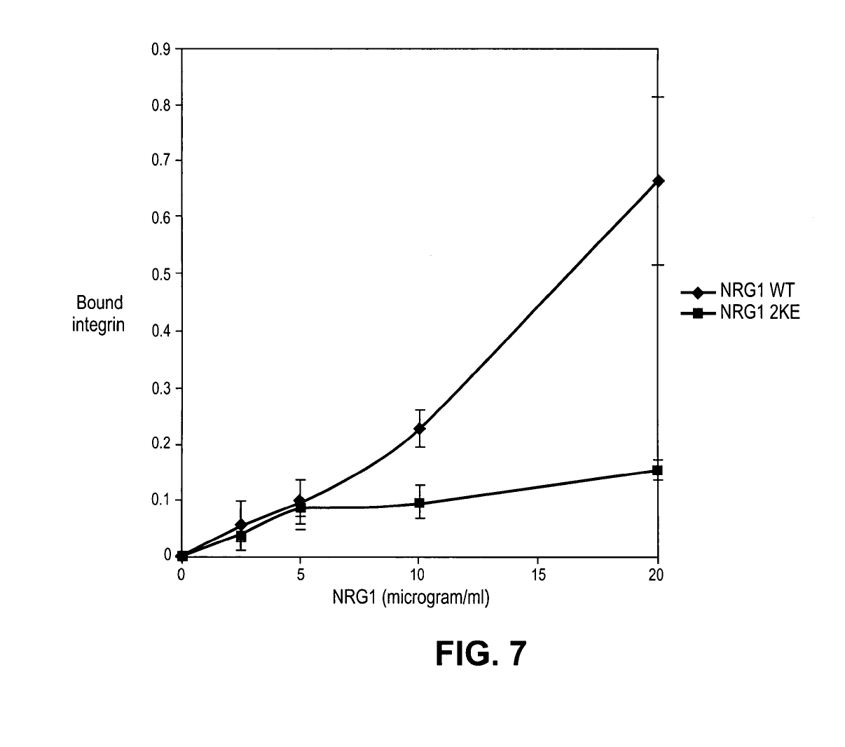

Docking simulation predicted the potential integrin-binding interface, which included Lys residues at positions 181, 185, and 187. NRG (175-222) mutants that do not bind to integrins were generated by mutating of Lys181/Lys185/Lys187 simultaneously to Glu residues (designated the 3KE (175-222) mutation) or Lys185/Lys187 to Glu (designated the 2KE (175-222) mutation). Both 2KE (175-222) (FIG. 7) and 3KE (175-222) mutants showed much lower affinity to αvβ3. The mutated Lys residues are located on the side opposite to the ErbB-binding site, indicating that the integrin-binding-defective mutants may interact with ErbB.

### Example 4

### NRG1 (175-222) Mutant Suppresses NRG1/ErbB Signaling in M21 Melanoma Cells

M21 cells express integrin αvβ3 at high levels and have been used for studying the role of this integrins. Human M21 melanoma cells were used because a variant of M21 that lacks αvβ3 expression (M21-L) is available (Cheresh and Spiro 1987). M21-L may be a good host for transfection of αvβ3 mutants in the future studies. However, M21 cells have not been widely used for studying ErbB signaling. ErbB2 and ErbB3 were detected, but not ErbB4, in M21 cells. NRG1 (175-222) was tested to induce the ErbB2/ErbB3 complex upon binding of NRG1 (175-222) to ErbB3.

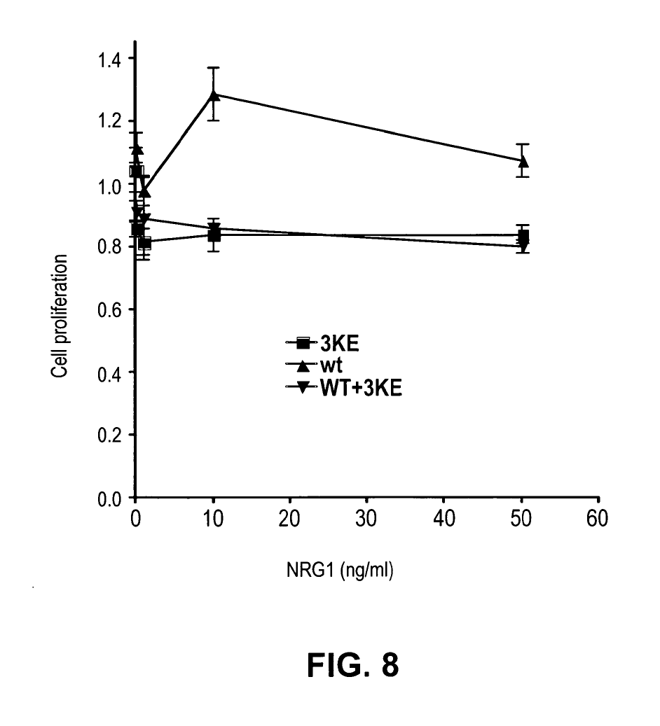

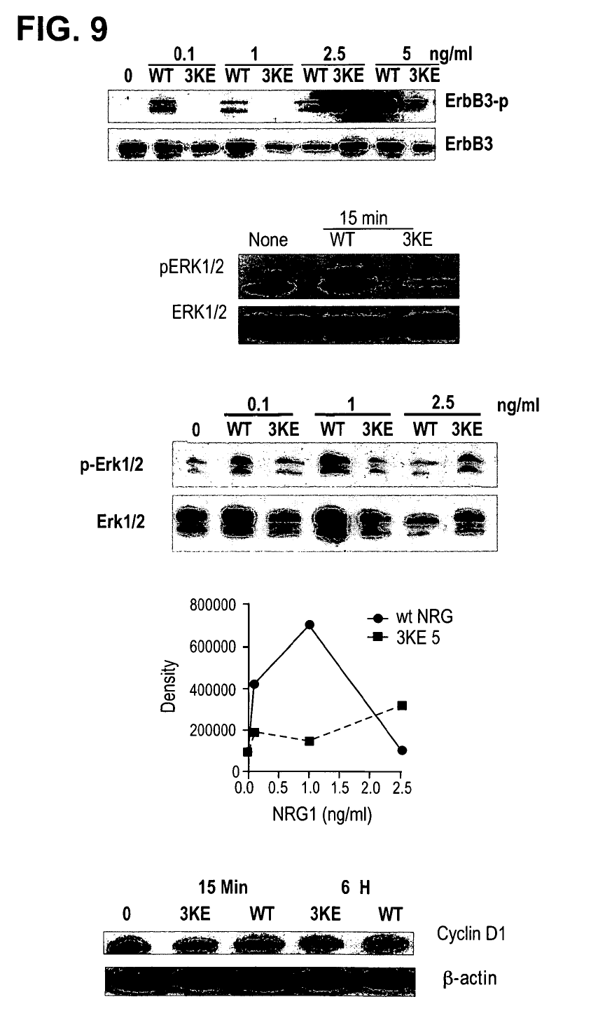

It was first tested whether the 3KE NRG1 (175-222) mutant would affect proliferation of M21 cells. The level of cell proliferation upon NRG1 (175-222) stimulation was measured by MTS assays. It was found that the 3KE (175-222) mutant did not induce proliferation of M21 melanoma cells, while wt NRG1 (175-222) did. It was also found that the 3KE (175-222) mutant suppressed the proliferation induced by wt NRG1 (175-222) when they are added together (FIG. 8). This suppression by 3KE (175-222) was a dominant-negative effect by definition. Activation of ERK1/2 was measured by western blotting. Consistent with the effect on cell proliferation, wt NRG1 (175-222) enhanced the level of ERK1/2 activation, and the 3KE (175-222) mutant did not induce the level of ERK1/2 activation (FIG. 9). It was further observed that cyclin D1 levels as a marker for cell cycle reduced with time. The ability to bind to integrins is believed critical for the mitogenic activity of NRG1 (175-222). When the levels of ERK1/2 activation and cyclin D1 levels were not completely suppressed after serum starvation, it appeared that the 3KE (175-222) mutant suppressed their levels.

### Example 5

### NRG1 (175-222) Mutant Suppresses NRG1/ErbB Signaling in Mouse B16F Melanoma Cells

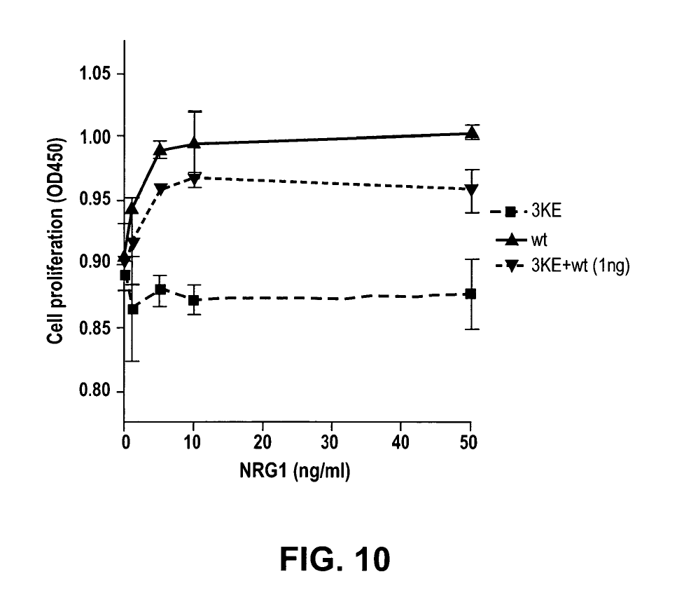

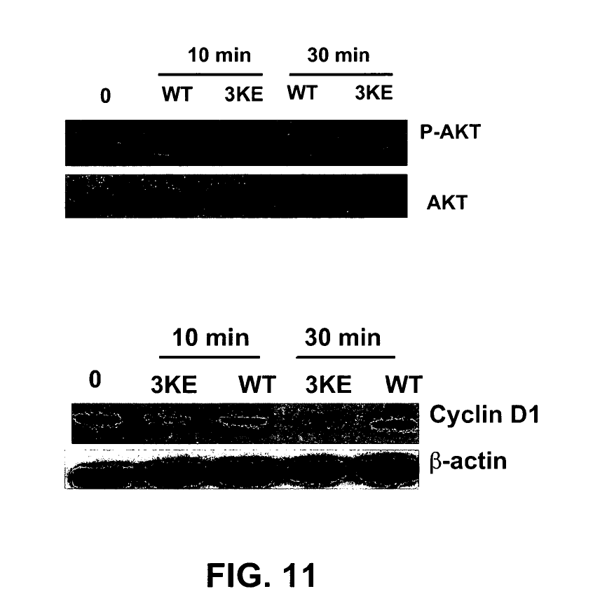

The mouse B16F10 melanoma xenograft model has been widely used for studying anti-cancer therapeutics. It was tested if this model is useful for studying the role of integrins in NRG1 signaling. B16F10 cells express ErbB2 and B3, but not ErbB4. It was found that wt NRG1 (175-222) induced cell proliferation (FIG. 10) and ERK1/2 and AKT activation (FIG. 11) in B16F10 cells, but 3KE (175-222) did not induce them. Interestingly, 3KE (175-222) suppressed D1 cyclin levels very rapidly. Taken together, these results indicate that 3KE (175-222) suppressed cell proliferation and ErbB signaling in B16F10 cells and the suppression of ErbB signaling by 3KE (175-222) is not cell-type specific.

Taken together, the 3KE (175-222) mutant was defective in inducing cell proliferation, ErbB3 phosphorylation, ERK1/2 or AKT activation, and suppressed the levels of cyclin D1. The 3KE (175-222) mutant suppressed cell proliferation in a dominant-negative manner. Thus the inhibitory effect of the 3KE (175-222) mutant is not specific to cell-types. It is believed that the defect of the 3KE (175-222) mutant is related to its defect in integrin binding

### Example 6

### Effects 3KE (175-222) and NRG1 (175-222) on Breast Cancer Cells

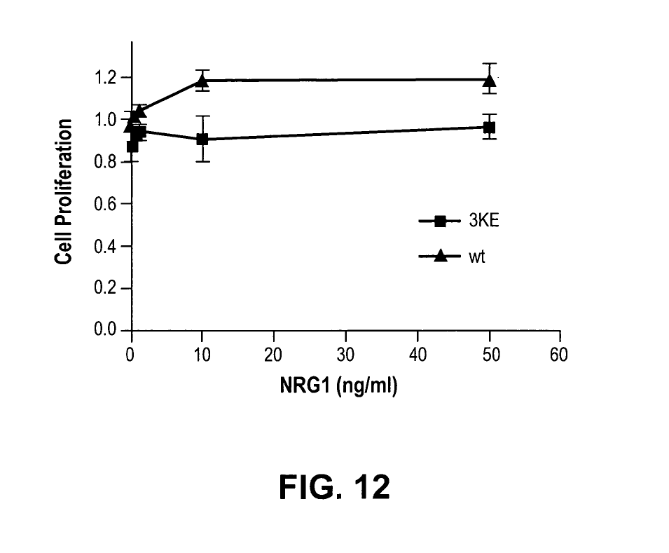

Several estrogen-dependent and independent breast cancer cell lines were tested and it was observed that wt NRG1 (175-222) enhanced the growth of breast cancer cells while the 3KE (175-222) mutant did not (FIG. 12 shows the results in MCF-7 cells as an example). Similar results were obtained with other breast cancer cell lines.

### Example 7

### NRG1 (175-222), But Not 3KE (175-222), Induced Tyr Phosphorylation of the Integrin β3 Cytoplasmic Domain

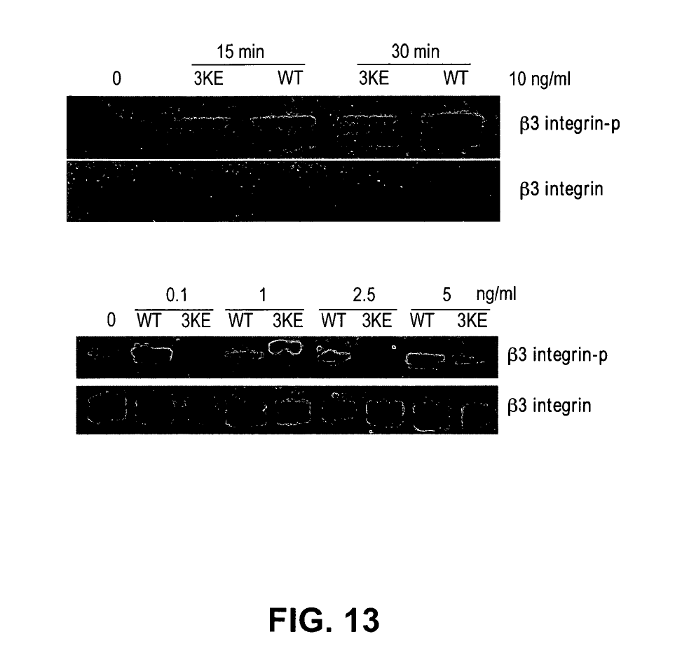

It has recently been reported that Tyr phosphorylation of the β3 tail plays a role in VEGF and insulin-like growth factor-1 (IGF1) (Clemmons et al. 2007; Mahabeleshwar et al. 2007). It was tested if NRG1 signaling requires Tyr-phosphorylation of the β3 tail in M21 cells. As shown here, the β3 tail is constitutively Tyr-phosphorylated in serum-starved cells. Wt NRG1 (175-222) enhanced levels of Tyr-phosphorylation in 30 min, but 3KE (175-222) suppressed it (FIG. 13).

### Example 8

### NRG1 (175-222) Suppressed ERK1/2, But 3KE (175-222) Enhanced ERK1/2 in an ErbB4-Dependent Manner

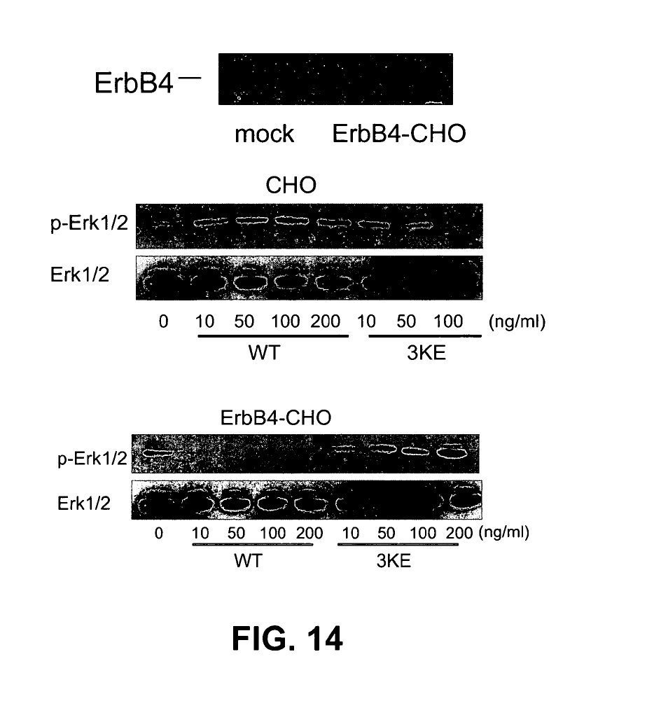

It has been reported that wt NRG1 suppressed ERK1/2 and PI-3K inhibitor blocked this suppression in an ErbB4-dependent manner using Chinese hamster ovary (CHO, ovarian cancer) cells that express recombinant human ErbB4 (Hatakeyama et al. 2003). CHO cells express endogenous ErbB2 and ErbB3, but no ErbB4. CHO cells that express ErbB4 were generated by transfecting ErbB4 expression vector (designated ErbB4-CHO). In mock-transfected CHO cells, wt NRG1 (175-222) enhanced ERK1/2 activation and 3KF (175-222) suppressed ERK1/2 activation, which is consistent with the previous report and the results using M21 melanoma. In contrast, in ErbB3-CHO cells, the 3KE (175-222) mutant activated ERKI/2 while wt NRG1 (175-222) suppressed ERK1/2 (FIG. 14). These results indicate that the 3KE mutation of NRG1 (175-222) blocked the suppression of ERK1/2 activation. It is therefore believed that the direct binding of integrin negatively regulates the Ras-MAP kinase pathway in ErbB4-CHO cells. Also, these results show that the 3KE (175-222) mutant is not defective in binding to ErbB4

### Example 9

### Effects of the 3KE (175-222) Mutant on Tumor Growth In Vivo

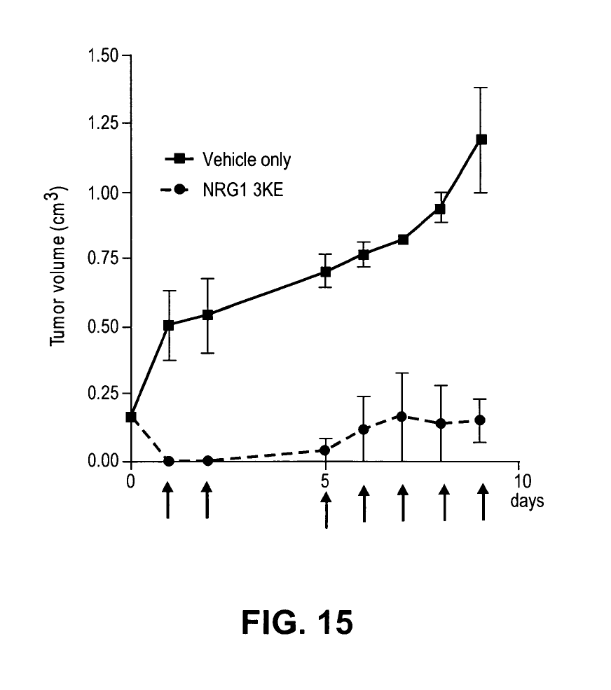

It was tested whether intraperitoneal injection of 3KE (175-222) can affect tumor growth in vivo. The Met-1 line (a highly metastatic mouse mammary tumor) (Guy et al. 1992; Cheung et al. 1997) has the polyoma virus middle T (PyV-MT) transgene, and metastasize with 100% efficiency. Met-1 (4mm×4 mm×4 mm) was transplanted to nude mice and 3KE (175-222) (100 ng/mouse/day 5 days a week) was intraperitoneally injected. It was observed that 3KE (175-222) markedly suppressed tumor growth at this low dose (FIG. 15).

### Example 10

### Inhibitory Effects of 3KE (175-222) on Pre-Malignant Cancer (MIN-O) In Vivo

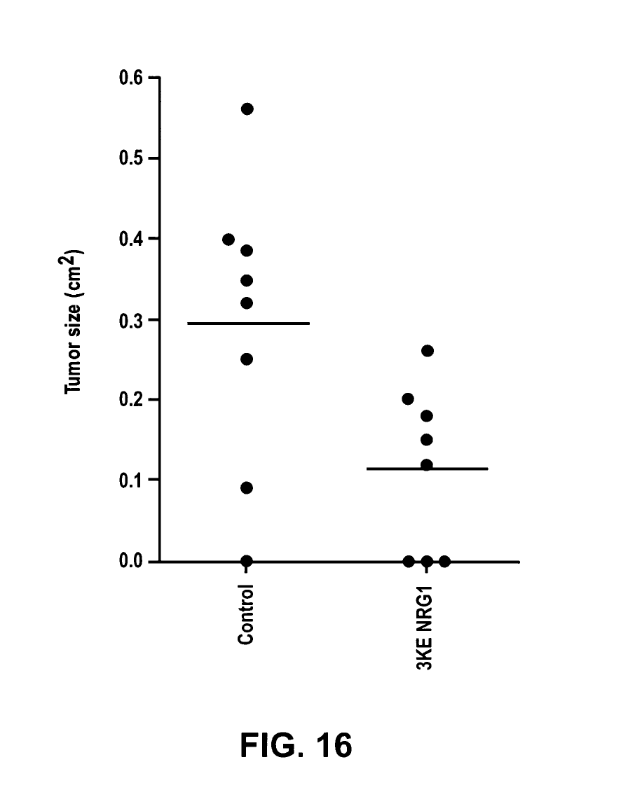

To study the effect of 3KE (175-222) on the premalignant cancer, transplantable mammary intraepithelial neoplasia-outgrowth (MIN-O) tissue lines, which were derived from hyperplastic mammary lesions in young Tg(MMTV-PyV-mT) females (Maglione et al. 2004), were used. The resulting lesions mimic the biological behavior, molecular biology, and histopathology of human ductal carcinoma in situ (Maglione et al. 2004). One mm³ pieces of the 8w-BMINO tissues (Maglione et al. 2004) were transplanted to gland-cleared no. 4 mammary fat pads of 3-week-old FVB females bilaterally. 3KE (175-222) (200 ng/day/mouse) was intraperitoneally injected 5 days a week for 4 weeks to FVB mice that had been transplanted with MIN-O. Control γC399tr did not affect the outgrowth of MIN-O. It was found that 3KE (175-222) effectively suppressed the outgrowth of MIN-O tumors after treatment for 4 week, indicating that blocking NRG signaling is effective in suppressing this pre-malignant legion outgrowth (FIG. 16).

Taken together, these in vivo results indicate that the 3KE (175-222) mutant can be used as an anti-cancer therapeutic, and that daily intraperitoneal injection is an effective way to deliver 3KE (175-222).

### Example 11

### NRG1 (175-222) Interacts with α6β4

Suppression of tumor growth by inhibition of ErbB receptor signaling is well documented. Relatively little is known about the ErbB signaling system in the regulation of angiogenesis, a process necessary for tumor growth. It has been reported that human umbilical code endothelial cells (HUVEC) highly express ErbB2, ErbB3, and ErbB4 and recombinant wt NRG1 mediates angiogenesis by a direct mechanism that stimulates endothelial cell functions (Russell et al. 1999). It has also been reported that NRG1 induces angiogenesis by indirect mechanisms through stimulation of VEGF expression from breast cancer cells (Bagheri-Yarmand et al. 2000; Yen et al. 2000; Xiong et al. 2001; Nakano et al. 2004). A recent paper reports that due to the lack of NRG1 receptors (ErbB3 and ErbB4) in several primary endothelial cell lines, NRG1 did not directly stimulate cellular responses in cultured endothelial cells (livanainen et al. 2007). This suggests that soluble NRG1 may not be a direct effecter for endothelial cells. It has been reported that epithelial cells or fibroblasts can be stimulated by NRGs and synthesize VEGF and other growth factors that affect endothelial cells and induce angiogenesis (Iivanainen et al. 2007). It has been shown that 3KE (175-222) markedly suppressed the growth of Met-1 tumor and MIN-O pre-malignant legion in vivo. It is therefore believed that this suppression includes direct effects of 3KE (175-222) on Met-1 or MIN-O and indirect effects through surrounding cells, including epithelial cells.

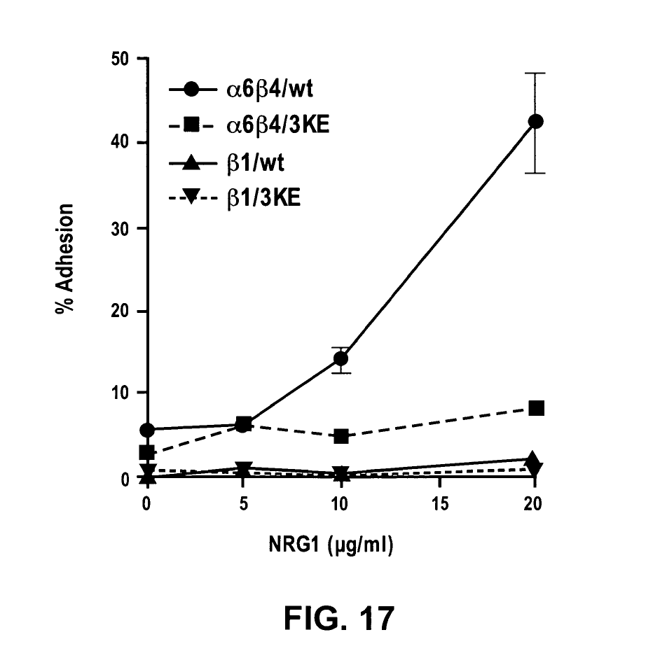

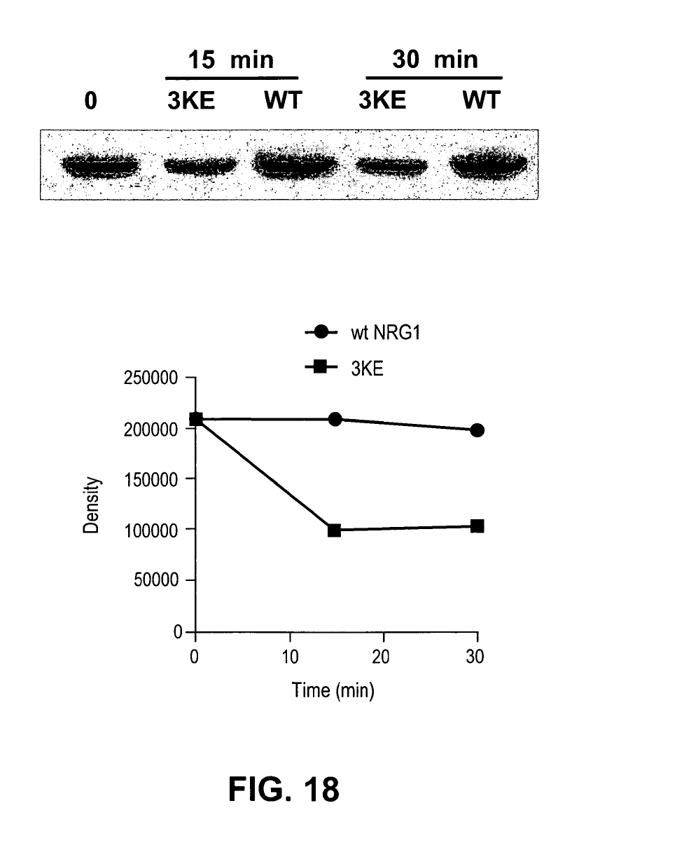

Because keratinocytes do not express αvβ3, it was expected that integrins other than αvβ3 may be involved in NRG1 signaling. CHO cells that express keratinocyte integrin α6β4 (α6β4-CHO) were observed to adhere to wt NRG1, but CHO cells that express human β1 did not. Interestingly, α6β4-CHO cells did not adhere to the 3KE (175-222) mutant (FIG. 17). These findings support the conclusion that NRG1 (175-222) is a ligand for α6β4 in keratinocytes, and 3KE (175-222) is defective in binding to a6134. It was further discovered that 3KE (175-222) suppressed levels of ERK1/2 activation (FIG. 18), demonstrating that 3KE (175-222) can suppress ErbB signaling in keratinocytes.

### Methods

Protein synthesis: Wt and mutant NRG1 (175-222) (EGF-like domains) were synthesized in E. coli as a GST fusion protein using pGEX-2T vector. Proteins were purified from cell extracts by glutathione affinity chromatography and then washed with 1% Triton X-114 to remove endotoxin before elution with 5 mM reduced glutathione. These proteins were used throughout the proposed experiments.

Cell proliferation: cells were starved by culturing DMEM with low-level FCS (e.g., 0.4%) overnight and then treated with the wt or mutant NRG1 (175-222) (up to 100 ng/ml). The cells were harvested in 15 min to 6 h after stimulation and cell lysates analyzed by western blotting with antibodies specific to signaling molecules. Alternatively, cells were cultured for 24-48 h after stimulation for testing proliferation or cell viability by MTS assays. Statistical analysis and calculation of kinetic constants were performed using Prism software (Graphpad).

Signaling: The levels of cell proliferation were measured by MTS assays and the kinetic data were analyzed by using Prism software (Graphpad). Melanoma cells were cultured in the presence of wt or mutant NRG1 (175-222), and the levels of ErbB2 and B3 phosphorylation, MAP kinase activation, or activation of other signaling molecules in the cell lysates were monitored by immunoprecipitating ErbB2 and B3 from the lysates and detecting tyrosine phosphorylation by western blotting with anti-phosphotyrosine antibodies (the IP-Western method) for ErbB2 and B3. Alternatively, anti-phospho ErbB2 or B3 antibodies (commercially available) and anti-phospho ERK1/2 antibodies were used.

Transfection: ErbB4 stably transfected cells were generated by transfecting ErbB4 expression plasmid pCDNA3.1 (with a neo gene) to cells and selected for G418 resistance. Western blotting analysis were performed as described above, and mutagenesis was carried out by the Quick-Change method.

CHO or K562 cells expressing different recombinant integrins were previously known and available (Zhang et al. 1998; Tarui et al. 2006). Labeling of proteins with FITC or Alexa480 was performed according to manufacturers' instructions. Flow cytometry was performed as described above. Also, adhesion assays as described above were used to confirm the findings using flow cytometry.

Examples 12-27 were carried out using wild type human neuregulin 1 fragments containing residues 175-241 of SEQ ID NO. 4, and mutants thereof. 3KE or 3KE (175-241) in Examples 12-27 has an amino acid sequence of SEQ ID NO:6. 2KE or 2KE (175-241) in Examples 12-27 has an amino acid sequence of SEQ ID NO:7.

### Example 12

### Antibodies, Recombinant Proteins, Recombinant Cells

Antibodies against phospho-ErbB3 (Tyr1289), phospho-Erk1/2 (Tyr 202 and Tyr 204), phosphor-Akt (Thr308), Erk1/2, Akt and Integrin (33 were purchased from Cell Signaling Technology, Inc. (Danvers, Mass.). Antibody against ErbB3 was purchased from Santa Cruz Biotechnology (Santa Cruz, Calif.). Horseradish peroxidase-conjugated anti-rabbit IgG was purchased from Bio-Rad Laboratories (Hercules, Calif.). Recombinant human ErbB3 Fc chimera was purchased from R&D Systems (Minneapolis, Minn.). Recombinant soluble αvβ3 and K562 cells that express human αvβ3 (αvβ3-K562) have been described (Saegusa, et al., 2008). Chinese hamster ovary (CHO) cells that express WT β1 or the β1-3-1 mutant have been described (Takagi, et al., 1997).

### Example 13

### Plasmid Construction

The GST-NRG1α (175-241) fusion protein used has the (GST)-GTSHLVKCAEKEKTFCVNGGECFMVKDLSNPSRYLCKCQPGFTGARCTENVPMKVQ NQEKAEELYQK sequence, which includes the entire EGF-like motif and the a domain. The cDNA fragment encoding the EGF-like domain was amplified using PCR with human NRG1 (the SMDF variant) cDNA (MGC-743, ATCC) as a template, and further extended to include the a domain by overlap extension PCR to include the entire sequence described above. A BamHI restriction site was introduced at the 5′ end and an EcoRI site at the 3′ end of the cDNA fragment. The resulting fragments were digested with BamHI and EcoRI and then subcloned into the BamHI/EcoRI sites of the pGEX-2T (Amersham Biosciences) vector. Site-directed mutagenesis was performed using the QuickChange method (Wang, et al., 1999). The presence of the mutations was verified by DNA sequencing.

### Example 14

### Protein Expression, and Purification of the WT and 3KE Mutant NRG1α (175-241)

The WT NRG1α (175-241) and its mutants were synthesized in E. coli BL21 (DE3) pLysS Rosseta gami 2 (Novagen) by inducing with 0.2 mM isopropyl β-D-thiogalactopyranoside for 2 h at room temperature. GST-NRG1α (175-241) was purified by glutathione affinity chromatography from bacterial extracts as described in the manufacturer's instructions (GE Healthcare, Piscataway, N.J.). To remove endotoxin, glutathione agarose column was extensively washed with 1% Triton X-114 in PBS before eluting proteins with 5 mM glutathione. The purified GST-fusion NRG1α (175-241) preparations were more than 90% homologous in SDS-PAGE and were kept in 1 mM reduced glutathione/2 mM oxidized glutathione in PBS at 4° C. to maintain disulfide bonds.

### Example 15

### Cell Culture

MCF-7 human breast cancer cells and Chinese hamster ovary (CHO) cells were cultivated in DMEM (GIBCO) supplemented with 10% (v/v) fetal bovine serum, 100 IU/ml penicillin, 100 μg/ml streptomycin, 0.25 μg/ml amphotericin B and non essential amino acid. K562 human erythroleukemia cells were cultivated in RPMI 1640 medium (GIBCO) supplemented with 10% (v/v) fetal bovine serum, 100 IU/ml penicillin, 100 μg/ml streptomycin, 0.25 μg/ml amphotericin B and non essential amino acid.

### Example 16

### Binding of Soluble αvβ3

Cell adhesion and soluble integrin binding assays were performed as described previously (Mori, et al., 2008). NRG1α (175-241) was immobilized to wells of 96-well microtiter plate overnight at 4° C. in 0.1 M carbonate buffer, pH 2 9.4. Remaining protein binding sites were blocked by incubating with 200 μl of 0.1% BSA in PBS for 60 min at room temperature. Wells were then incubated with soluble integrin αvβ3 in 50 μl in Hepes-Tyrode's buffer supplemented with 1 mM Mn2+ at room temperature for 60 min. After rinsing the wells with the same buffer, bound integrins were determined by horseradish peroxidase (HRP)-conjugated anti-His tag mouse IgG and substrate 3,3′,5,5′-tetramethylbenzidine of HRP.

### Example 17

### Competitive Binding Assay

GST-fusion WT NRG1α (175-241) was biotinylated by using EZ-Link Sulfo-NHS-LC-Biotin (Pierce) as described in the manufacturer's instructions. Briefly, GST-fusion WT NRG1α (175-241) was incubated with sulfo-NHS-LCBiotin for 1 hour on ice, and remaining free sulfo-NHS-LC-Biotin was quenched with Tris-HCl buffer pH 8.0. Recombinant human ErbB3 Fc chimera (R&D Systems) was immobilized to wells of 96-well microtiter plate at 1 μg/ml coating concentration in 0.1 M NaHCO3, pH9.4 overnight at 4° C., and the remaining protein binding sites were blocked by incubating with 0.1% BSA. Wells were then incubated with biotinylated GST fusion NRG1α (175-241) in the presence of non-labeled GST, GST-WT NRG1α (175-241) or GST-3KE (175-241) for 3 h at room temperature. Bound biotinylated GST-fusion NRG1α (175-241) WT to wells was determined with streptavidin HRP conjugate and HRP substrate at 490 nm.

### Example 18

### Proliferation Assay

MCF-7 cells (1×103) were serum-starved overnight in serum-free DMEM and then stimulated with WT or mutant NRG1α (175-241) for 48 h. Cell proliferation was measured using MTS assays.

### Example 19

### Western Blot Analysis

MCF-7 cells grown to confluence were serum-starved in serum-free medium overnight, and then treated with WT or 3KE mutant NRG1α (175-241) (2.5 nM) for 5 min to 6 h at 37° C. Cells were washed with ice-cold PBS once, and lysed with the lysis buffer (20 mM HEPES (pH 7.4), 100 mM NaCl, 10% Glycerol, 1% NP-40, 1 mM MgCl2, 5 mM EDTA, 0.5% Triton X-100, 1 mM phenylmethylsulfonyl fluoride, 20 mM NaF, 1 mM Na3VO4, protease inhibitor cocktail (Sigma-Aldrich)). Protein concentrations in the cell lysates were determined using BCA protein assay (Pierce). Equal amounts of cell proteins were analyzed by SDS-PAGE and transferred onto 0.45 μm pore-size polyvinylidene fluoride membrane (Milipore, Birellica, Mass.). The membrane was incubated with primary antibodies, then HRP-conjugated secondary antibody and enhanced chemiluminescence detection reagents (Pierce).

### Example 20

### Co-Immunoprecipitation

Five minutes to 3 h after treatment with 5 nM WT or 3KE mutant NRG1α (175-241), MCF-7 cells were washed with ice-cold PBS and lysed with lysis buffer. The cell lysate was incubated with anti-ErbB3 overnight at 4° C. The immune complex was recovered by incubating with protein A Sepharose (GE Healthcare) for 1 hour at 4° C. and washed three times with wash buffer (20 mM HEPES (pH 7.4), 100 mM NaCl, 10% Glycerol, 0.5% NP-40, 1 mM MgCl2, 5 mM EDTA, 0.5% Triton X-100, 1 mM phenylmethylsulfonyl fluoride, 20 mM NaF, 1 mM Na3VO4, protease inhibitor cocktail (Sigma-Aldrich)). The immunoprecipitates were analyzed by western blotting with antibodies specific to integrin β3.

### Example 21

### Other Methods

Docking simulation was performed as previously described (Saegusa, et al., 2008; and Mori, et al., 2008), using AutoDock3 and ADT (Sanner, 1999). In the present invention, PMV 1.54 (Sanner, 1999) was used for graphics and Swiss-pdb viewer 4.01 (Swissprot) for superposing TGFα and NRG1 (175-241).

### Example 22

### Direct Binding of Integrin αvβ3 to NRG1α (175-241)

In the present invention, it was tested if soluble recombinant integrin αvβ3 binds to immobilized GST-NRG1α (175-241) (ELISA-type assay). In the present invention, it was found that soluble αvβ3 integrin bound to GST-NRG1α (175-241) (isolated EGF-like domain) in a dose-dependent manner.

In the present invention, it was also tested if cell-surface integrins bind to immobilized NRG1α (175-241). Chinese hamster ovary (CHO) cells that express recombinant β3 (β3-CHO) adhered to NRG1α (175-241), while CHO cells that express 3 recombinant human β1 (β1-CHO) did not.

K562 erythroleukemic cells expressing recombinant αvβ3 (αvβ3-K562) adhered in a dose-dependent manner, but mock-transfected K562 cells did not. These results indicate that the EGF-like domain of NRG1α (175-241) directly interacts with integrin αvβ3. Furthermore, anti-β3 mAb (7E3) and cyclic RGDfV (an-αvβ3- specific antagonist) reduced the adhesion. These results indicate that binding of NRG1α (175-241) to αvβ3 is specific.

The integrin β subunit possesses an I-like domain that plays a critical role in ligand binding (Luo, 2007). In the present invention, it has been shown that when a disulfide-linked five-residue sequence of β1 I-like domain (residues 177-183) of αvβ1 is switched with a corresponding sequence in β3 integrin (designated the β1-3-1 mutant), ligand-binding specificity of the mutated integrin αvβ1-3-1 is altered to that of αvβ3 (Takagi, et al., 1997). Hence the loop was designated “the specificity loop”. The β1-3-1 mutant (as αvβ1-3-1) bound to vitronectin and fibrinogen, but wt β1 (as αvβ1) did not (Takagi, et al., 1997). The crystal structure of αvβ3 showed that the specificity loop is located in the ligand-binding (RGD-binding) site and undergoes marked conformational changes (1 angstrom shift) upon RGD binding to αvβ3 (Xiong, et al. 2002). To determine if NRG1α (175-241) binds to the ligand-binding site of αvβ3 common to other known αvβ3 ligands (e.g., vitronectin and fibrinogen), In the present invention, the β1-3-1 mutant (Takagi, et al., 1997) was used. CHO cells that express β1-3-1 (designated β1-3-1-CHO cells) bound to NRG1α (175-241), but wt β1l-CHO did not. The adhesion of β1-3-1-CHO cells to NRG1α (175-241) was blocked by anti-β1 antibody AIIB2 (Note: the β1-3-1mutant is still more than 99% human β1, and therefore its function is blocked by anti-human β1 mAb such as AIIB2 (Takagi, et al., 1997). These findings indicate that NRG1α binds to the ligand-binding site of αvβ3, and that the specificity loop plays a role in NRG1α binding to αvβ3.

### Example 23

### Integrin-Binding-Defective Mutant of NRG1α (175-241)

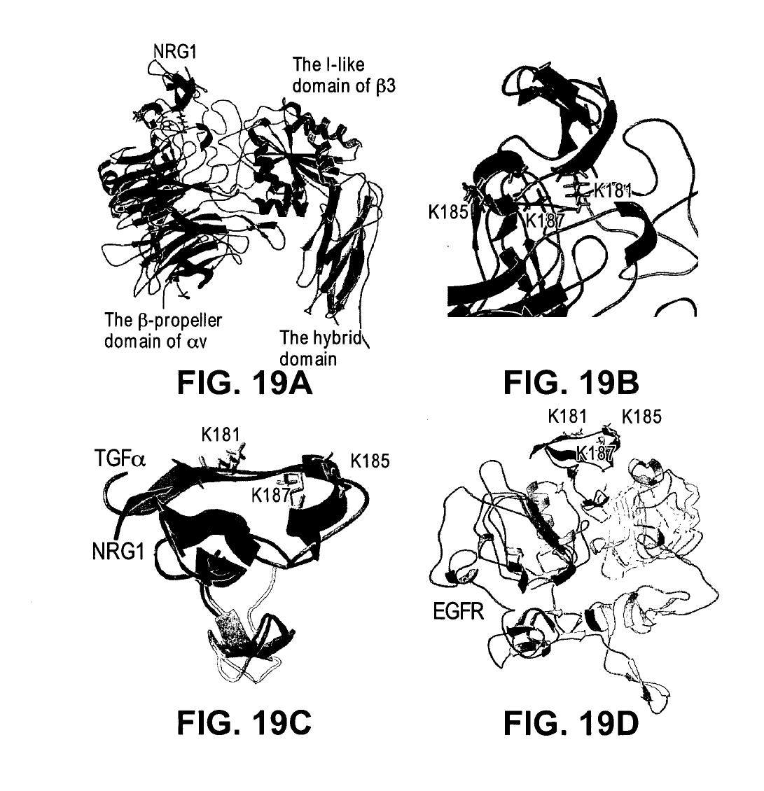

To locate the integrin-binding site in NRG1α and to generate integrin-binding-defective mutants of NRG1α, in the present invention, docking simulation and site-directed mutagenesis were used. Docking simulation of the interaction between integrin αvβ3 and the EGF-like domain of NRG1α predicts that NRG1α binds to integrin αvβ3 with a high affinity (docking energy −23.5 kcal/mol) (FIG. 19a), which is consistent with our binding results. The predicted integrin-binding interface includes the Lys residues at positions 181, 185, and 187 (FIG. 19b), which are conserved in NRG1α and NRG1β. To assess the position of the Lys residues in the NRG1-ErbB complex, in the present invention, the TGFα-EGFR complex, PDB code 1MOX was used, since NRG1 and TGFα are homologous (FIG. 19c). In the present invention, the TGFα was replaced by. NRG1α, by superposing them (FIG. 19d). The model predicted that the Lys residues at positions 181, 185, and 187 are not in the EGFR binding site of NRG1α.

In the present invention, the Lys residues were mutated simultaneously to Glu (designated the Lys181/Lys185/Lys187 to Glu (3KE (175-241) mutation). In the present invention, it was tested to see if the 3KE (175-241) mutant is defective in binding to integrin αvβ3 in cell adhesion assays using αvβ3-K562, control mock-transfected K562 cells, and β1-3-1-CHO cells. In the present invention, it was found that αvβ3 and αvβ1-3-1 mutant integrins bound to wt NRG1α (175-241). But there was little or no adhesion to the 3KE (175-241) mutant, indicating that the 3KE (175-241) mutant is defective in binding to αvβ3. In the present invention, similar results were obtained using β3-CHO cells.

### Example 24

### The 3KE (175-241) Mutant Binds to ErbB3

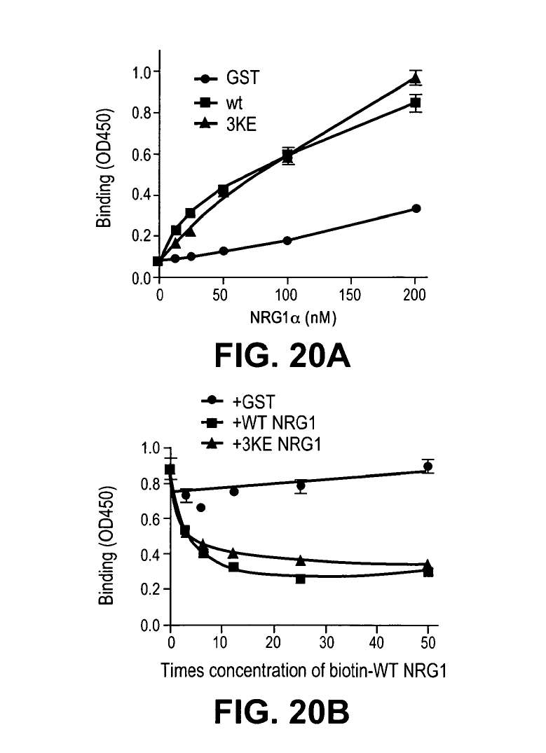

The docking simulation and our model (FIG. 19d) predict that the integrin-binding site in NRG1α is distinct from the ErbB binding site. In the present invention, it was tested to see if the 3KE mutation affects the binding of NRG1α to ErbB3 using recombinant soluble ErbB3. In the present invention, it was demonstrated that WT and 3KE mutant NRG1α (175-241) bound to immobilized soluble ErbB3 comparably in an ELISA-type assay (FIG. 20a). Also, In the present invention, WT and 3KE NRG1α (175-241) were shown to compete for binding of biotinylated WT NRG1α (175-241) to immobilized soluble ErbB3 at comparable levels in a competitive binding assays (FIG. 20b). These findings suggest that the 3KE mutation has minimal effects on NRG1α-ErbB3 interaction.

### Example 25

### NRG1α (175-241) Induces Co-Precipitation of αvβ3 and ErbB3, while 3KE (175-241) is Defective in this Function

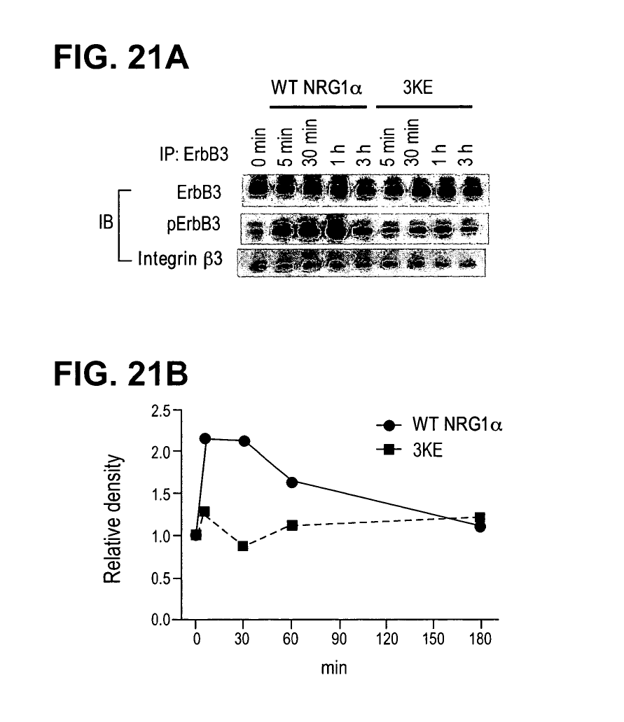

If NRG1α binds to both ErbB3 and integrin αvβ3, it is predicted that NRG1α mediates ternary complex (αvβ3-NRG1α-ErbB3) formation. In the present invention, it was tested this possibility using MCF-7 human breast cancer. MCF-7 was chosen since NRG1 stimulation of MCF-7 cells induces intracellular signaling (e.g., AKT activation) via signaling of ErbB2-ErbB3 heterodimers (Liu, et al., 1999). In the present invention, it was demonstrate that stimulation with WT NRG1α (175-241) increased the amount of integrin β3 protein that co-precipitates with ErbB3 in 5-30 min in MCF-7 cells, while the 4 stimulation with 3KE (175-241) did not (FIGS. 21a and 21b). The levels of ErbB3 were comparable throughout the incubation period. These results suggest that NRG1α induces ErbB-NRG1 integrin complex formation, and this process is dependent on the ability of NRG1α to interact with integrin. The integrin β3 subunit is highly Tyr phosphorylated in MCF-7 cells (data not shown), and thus it is unclear if Tyr phosphorylation of β3 is induced by NRG1α or if this process is required for NRG1α signaling.

### Example 26

### The 3KE (175-241) Mutant is Defective in Inducing Intracellular Signaling

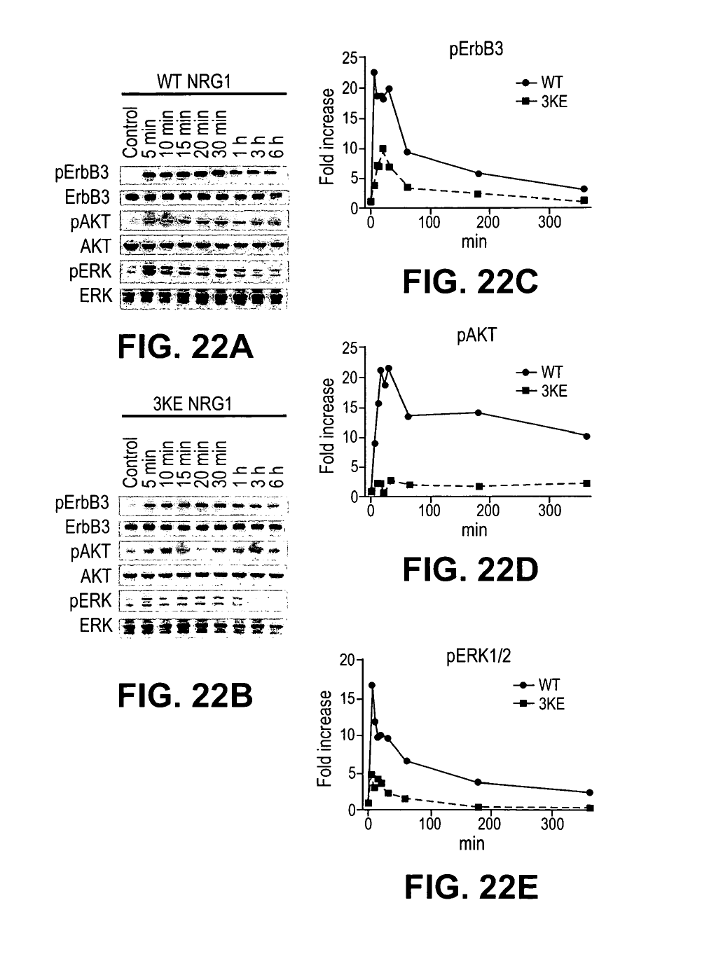

Taken together, in the present invention, it was demonstrated that 1) NRG1α binds to αvβ3 in a way predicted by docking simulation, 2) the Lys residues in the predicted integrin-binding site are critical for integrin binding, 3) the 3KE (175-241) mutation is defective in integrin binding but minimally affects NRG1α (175-241) binding to ErbB3, and 4) NRG1α (175-241) induces co-precipitation of ErbB3 and αvβ3 and this process is dependent on the ability of NRG la to bind to integrin. These results indicate that the ability of NRG1α to interact with αvβ3 is essential for NRG1/ErbB signaling. In the present invention, it was tested to see if the 3KE (175-241) mutation affects the ability to induce NRG1α (175-241) intracellular signaling in MCF-7 cells. In the present invention, it was found that the 3KE (175-241) induced ErbB3 phosphorylation, ERK1/2 activation at much lower levels than WT NRG1α (175-241) in MCF-7 cells (FIG. 22a-e). Notably, the 3KE (175-241) mutant induced little or no AKT activation (FIG. 22d). These results indicate that the 3KE (175-241) mutant is defective in inducing NRG1/ErbB intracellular signaling.

### Example 27

### Effect of 3KE NRG1 (175-241) Mutant on In Vivo Tumorigenesis

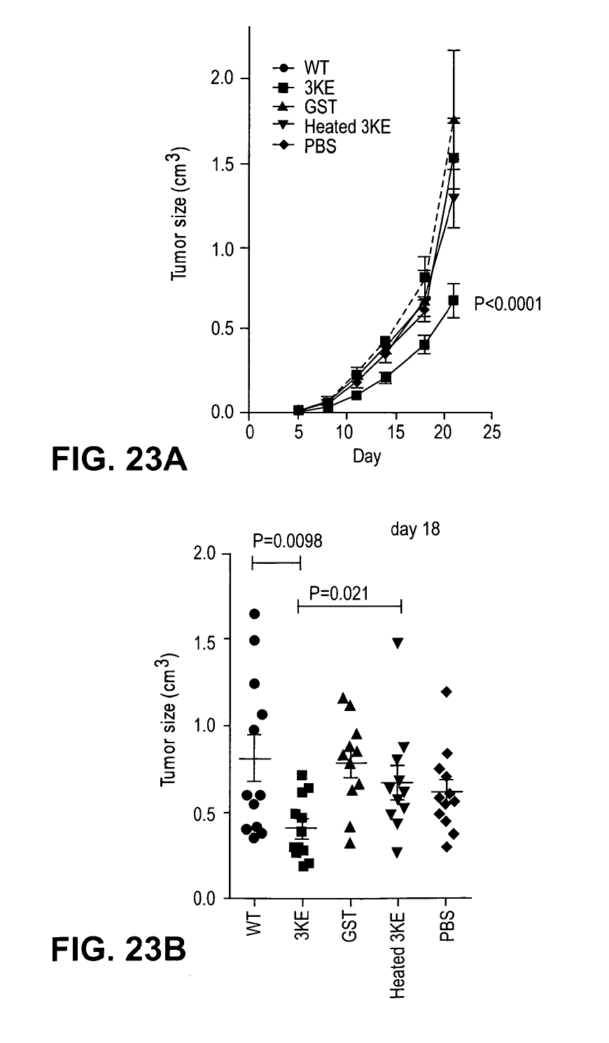

In the present invention, Met-1 mouse breast cancer fragment (2×2×2 mm) was transplanted to fat pad of FVB syngeneic mice (2 per animal, 5-6 mice per group) (day 0). At day 5, intraperitoneal injection of 200 ng/mouse/day wt NRG1, 3KE, heat-denatured 3KE or vehicle (PBS) everyday were started (FIG. 23). PBS contains 0.1 mg/ml mouse serum albumin as carrier. Tumor size was measured using caliper twice a week (FIG. 23). Tumor size was calculated using V=0.4×(a×b×b) where a is the longest diameter and b is a diameter perpendicular to a. Statistical analysis was performed using Prism software package.

All patents, patent applications, and other publications, including GenBank Accession Numbers, cited in this application are incorporated by reference in the entirety for all purposes.

### REFERENCES

Akakura, N., C. Hoogland, Y. K. Takada, J. Saegusa, X. Ye, F. T. Liu, A. T. Cheung and Y. Takada (2006). “The COOH-terminal globular domain of fibrinogen gamma chain suppresses angiogenesis and tumor growth.” Cancer Res 66(19): 9691-7.
Alimandi, M., A. Romano, M. C. Curia, R. Muraro, P. Fedi, S. A. Aaronson, P. P. Di Fiore and M. H. Kraus (1995). “Cooperative signaling of ErbB3 and ErbB2 in neoplastic transformation and human mammary carcinomas.” Oncogene 10(9): 1813-21.
Alroy, I. and Y. Yarden (1997). “The ErbB signaling network in embryogenesis and oncogenesis: signal diversification through combinatorial ligand-receptor interactions.” FEBS Lett 410(1): 83-6.
Amin, D. N., K. Hida, D. R. Bielenberg and M. Klagsbrun (2006). “Tumor endothelial cells express epidermal growth factor receptor (EGFR) but not ErbB3 and are responsive to EGF and to EGFR kinase inhibitors.” Cancer Res 66(4): 2173-80.
Atlas, E., M. Cardillo, I. Mehmi, H. Zahedkargaran, C. Tang and R. Lupu (2003). “Heregulin is sufficient for the promotion of tumorigenicity and metastasis of breast cancer cells in vivo.” Mol Cancer Res 1(3): 165-75.
Bagheri-Yarmand, R., R. K. Vadlamudi, R. A. Wang, J. Mendelsohn and R. Kumar (2000). “Vascular endothelial growth factor up-regulation via p21-activated kinase-1 signaling regulates heregulin-betal-mediated angiogenesis.” J Biol Chem 275(50): 39451-7.
Berger, M. S., G. W. Locher, S. Saurer, W. J. Gullick, M. D. Waterfield, B. Groner and N. E. Hynes (1988). “Correlation of c-erbB-2 gene amplification and protein expression in human breast carcinoma with nodal status and nuclear grading.” Cancer Res 48(5): 1238-43.
Bergh, J. J., H. Y. Lin, L. Lansing, S. N. Mohamed, F. B. Davis, S. Mousa, and P. J. Davis, Integrin alphaVbeta3 contains a cell surface receptor site for thyroid hormone that is linked to activation of mitogen-activated protein kinase and induction of angiogenesis. Endocrinology, 2005. 146(7): p. 2864-71.
Bon, G., V. Folgiero, S. Di Carlo, A. Sacchi, and R. Falcioni, Involvement of alpha6beta4 integrin in the mechanisms that regulate breast cancer progression. Breast Cancer Res, 2007. 9(1): p. 203.
Breuleux, M. (2007). “Role of heregulin in human cancer.” Cell Mol Life Sci 64(18): 2358-77.
Carpenter, G. (2003). “ErbB-4: mechanism of action and biology.” Exp Cell Res 284(1): 66-77.
Carraway, K. L., 3rd and L. C. Cantley (1994). “A neu acquaintance for erbB3 and erbB4: a role for receptor heterodimerization in growth signaling.” Cell 78(1): 5-8.
Carraway, K. L., 3rd, S. P. Soltoff, A. J. Diamonti and L. C. Cantley (1995). “Heregulin stimulates mitogenesis and phosphatidylinositol 3-kinase in mouse fibroblasts transfected with erbB2/neu and erbB3.” J Biol Chem 270(13): 7111-6.
Carraway, K. L., 3rd, Weber, J. L., Unger, M. J., Ledesma, J., Yu, N., Gassmann, M., and Lai, C. (1997) Nature 387, 512-516.
Chang, H., Riese, D. J., 2nd, Gilbert, W., Stern, D. F., and McMahan, U. J. (1997) Nature 387, 509-512.
Cheresh, D. A., R. Pytela, M. D. Pierschbacher, F. G. Klier, E. Ruoslahti and R. A. Reisfeld (1987). “An Arg-Gly-Asp-directed receptor on the surface of human melanoma cells exists in an divalent cation-dependent functional complex with the disialoganglioside GD2.” J. Cell Biol. 105(3): 1163-73.
Cheresh, D. A. and R. C. Spiro (1987). “Biosynthetic and functional properties of an Arg-Gly-Asp-directed receptor involved in human melanoma cell attachment to vitronectin, fibrinogen, and von Willebrand factor.” J. Biol. Chem. 262(36): 17703-11.
Cheung, A., L. Young and P. Chen (1997). “Microcirculation and metastasis in a new mouse mammary tumor model system.” Int. J. Oncol. 11: 69-77.
Clemmons, D. R., L. A. Maile, Y. Ling, J. Yarber and W. H. Busby (2007). “Role of the integrin alphaVbeta3 in mediating increased smooth muscle cell responsiveness to IGF-I in response to hyperglycemic stress.” Growth Horm IGF Res 17(4): 265-70.
Cordes, N., J. Seidler, R. Durzok, H. Geinitz, and C. Brakebusch (2006). “Beta1-integrin-mediated signaling essentially contributes to cell survival after radiation-induced genotoxic injury.” Oncogene 25(9):1378-90.
De Potter, I. Y., Y. Poumay, K. A. Squillace and M. R. Pittelkow (2001). “Human EGF receptor (HER) family and heregulin members are differentially expressed in epidermal keratinocytes and modulate differentiation.” Exp Cell Res 271(2): 315-28.
Elenius, K., C. J. Choi, S. Paul, E. Santiestevan, E. Nishi and M. Klagsbrun (1999). “Characterization of a naturally occurring ErbB4 isoform that does not bind or activate phosphatidyl inositol 3-kinase.” Oncogene 18(16): 2607-15.
Elices, M. J., L. Osborn, Y. Takada, C. Crouse, S. Luhowskyj, M. E. Hemler and R. R. Lobb (1990). “VCAM-1 on activated endothelium interacts with the integrin VLA-4 on leukocytes at a site distinct from the VLA-4/fibronectin binding site.” Cell 60: 577-584.
Erickson, S. L., O'Shea, K. S., Ghaboosi, N., Loverro, L., Frantz, G., Bauer, M., Lu, L. H., and Moore, M. W. (1997) Development 124, 4999-5011.
Eto, K., C. Huet, T. Tarui, S. Kupriyanov, H. Z. Liu, W. Puzon-McLaughlin, X. P. Zhang, D. Sheppard, E. Engvall and Y. Takada (2002). “Functional classification of ADAMs based on a conserved motif for binding to integrin in alpha 9beta 1; implications for sperm-egg binding and other cell interactions.” J. Biol. Chem. 277: 17804-17810.
Eto, K., W. Puzon-McLaughlin, D. Sheppard, A. Sehara-Fujisawa, X. P. Zhang and Y. Takada (2000). “RGD-independent binding of integrin alpha9beta1 to the ADAM-12 and -15 disintegrin domains mediates cell-cell interaction.” J. Biol. Chem. 275(45): 34922-30.
Falcioni, R., A. Antonini, P. Nistico, S. Di Stefano, M. Crescenzi, P. G. Natali and A. Sacchi (1997). “Alpha 6 beta 4 and alpha 6 beta 1 integrins associate with ErbB-2 in human carcinoma cell lines.” Exp. Cell Res. 236(1): 76-85.
Falls, D. L. (2003). “Neuregulins: functions, forms, and signaling strategies.” Exp Cell Res 284(1): 14-30.
Folgiero, V., R. E. Bachelder, G. Bon, A. Sacchi, R. Falcioni and A. M. Mercurio (2007). “The alpha6beta4 integrin can regulate ErbB-3 expression: implications for alpha6beta4 signaling and function.” Cancer Res 67(4): 1645-52.
Gambaletta, D., A. Marchetti, L. Benedetti, A. M. Mercurio, A. Sacchi and R. Falcioni (2000). “Cooperative signaling between alpha(6)beta(4) integrin and ErbB-2 receptor is required to promote phosphatidylinositol 3-kinase-dependent invasion.” J Biol Chem 275(14): 10604-10.
Garrett, T. P., N. M. McKern, M. Lou, T. C. Elleman, T. E. Adams, G. O. Lovrecz, M. Kofler, R. N. Jorissen, E. C. Nice, A. W. Burgess and C. W. Ward (2003). “The crystal structure of a truncated ErbB2 ectodomain reveals an active conformation, poised to interact with other ErbB receptors.” Mol Cell 11(2): 495-505.
Graef, T., U. Steidl, W. Nedbal, U. Rohr, R. Fenk, R. Haas, and R. Kronenwett, Use of RNA interference to inhibit integrin subunit alphaV-mediated angiogenesis. Angiogenesis, 2006: p. 1-12.
Guy, C. T., R. D. Cardiff and W. J. Muller (1992). “Induction of mammary tumors by expression of polyomavirus middle T oncogene: a transgenic mouse model for metastatic disease.” Mol. Cell. Biol. 12(3): 954-61.
Harari, D. and Y. Yarden, Molecular mechanisms underlying ErbB2/HER2 action in breast cancer. Oncogene, 2000. 19(53): p. 6102-14.
Hatakeyama, M., S. Kimura, T. Naka, T. Kawasaki, N. Yumoto, M. Ichikawa, J. H. Kim, K. Saito, M. Saeki, M. Shirouzu, S. Yokoyama and A. Konagaya (2003). “A computational model on the modulation of mitogen-activated protein kinase (MAPK) and Akt pathways in heregulin-induced ErbB signalling.” Biochem J 373(Pt 2): 451-63.
Hemler, M. E., C. Huang, Y. Takada, L. Schwarz, J. L. Strominger and M. L. Clabby (1987). “Characterization of the cell surface heterodimer VLA-4 and related peptides.” J. Biol. Chem. 262(24): 11478-85.
Hijazi, M. M., E. W. Thompson, C. Tang, P. Coopman, J. A. Toni, D. Yang, S. C. Mueller and R. Lupu (2000). “Heregulin regulates the actin cytoskeleton and promotes invasive properties in breast cancer cell lines.” Int J Oncol 17(4): 629-41.
Holbro, T., R. R. Beerli, F. Maurer, M. Koziczak, C. F. Barbas, 3rd and N. E. Hynes (2003). “The ErbB2/ErbB3 heterodimer functions as an oncogenic unit: ErbB2 requires ErbB3 to drive breast tumor cell proliferation.” Proc Natl Acad Sci USA 100(15): 8933-8.
Holbro, T., G. Civenni and N. E. Hynes (2003). “The ErbB receptors and their role in cancer progression.” Exp Cell Res 284(1): 99-110.
Holmes, W. E., M. X. Sliwkowski, et al. (1992). “Identification of heregulin, a specific activator of p185erbB2.” Science 256(5060): 1205-1210.
Hynes, N. E. and H. A. Lane (2005). “ERBB receptors and cancer: the complexity of targeted inhibitors.” Nat Rev Cancer 5(5): 341-54.
Hynes, R. O. (2002). “Integrins: bidirectional, allosteric signaling machines.” Cell 110(6): 673-87.
Iivanainen, E., I. Paatero, S. M. Heikkinen, T. T. Junttila, R. Cao, P. Klint, P. M. Jaakkola, Y. Cao and K. Elenius (2007). “Intra- and extracellular signaling by endothelial neuregulin-1.” Exp Cell Res.
Kamata, T. and Y. Takada (2001). “Platelet integrin alphaIIbbeta3-ligand interactions: what can we learn from the structure'?” Int. J. Hematol. 74(4): 382-9.
Karey, K. P., T. L. Riss, B. D. Burleigh, D. Parker and D. A. Sirbasku (1988). “Human recombinant insulin-like growth factor I. II. Binding characterization and radioreceptor assay development using Balb/c 3T3 mouse embryo fibroblasts.” In Vitro Cell Dev Biol 24(11): 1107-13.
Kim, H. and W. J. Muller (1999). “The role of the epidermal growth factor receptor family in mammary tumorigenesis and metastasis.” Exp Cell Res 253(1): 78-87.
Lemmens, K Doggen, K., and De Keulenaer, G. W. (2007) Circulation 116, 954-960.
Lemoine, N. R., D. M. Barnes, D. P. Hollywood, C. M. Hughes, P. Smith, E. Dublin, S. A. Prigent, W. J. Gullick and H. C. Hurst (1992). “Expression of the ERBB3 gene product in breast cancer.” Br J Cancer 66(6): 1116-21.
Li, Q., S. Ahmed and J. A. Loeb (2004). “Development of an autocrine neuregulin signaling loop with malignant transformation of human breast epithelial cells.” Cancer Res 64(19): 7078-85.
Ling, Y., L. A. Maile, J. Badley-Clarke and D. R. Clemmons (2005). “DOK1 mediates SHP-2 binding to the alphaVbeta3 integrin and thereby regulates insulin-like growth factor I signaling in cultured vascular smooth muscle cells.” J. Biol. Chem. 280(5): 3151-8.
Lipscomb, E. A., K. J. Simpson, S. R. Lyle, J. E. Ring, A. S. Dugan, and A. M. Mercurio, The alpha6beta4 integrin maintains the survival of human breast carcinoma cells in vivo. Cancer Res., 2005. 65(23): p. 10970-6.
Liu, W., Li, J., and Roth, R. A. (1999) Biochem Biophys Res Commun 261, 897-903.
Luo, B. H., C. V. Carman and T. A. Springer (2007). “Structural Basis of Integrin Regulation and Signaling.” Annu. Rev. Immunol.
Maglione, J. E., E. T. McGoldrick, L. J. Young, R. Namba, J. P. Gregg, L. Liu, D. Moghanaki, L. G. Ellies, A. D. Borowsky, R. D. Cardiff and C. L. MacLeod (2004). “Polyomavirus middle T-induced mammary intraepithelial neoplasia outgrowths: single origin, divergent evolution, and multiple outcomes.” Mol Cancer Ther 3(8): 941-53.
Mahabeleshwar, G. H W. Feng, K. Reddy, E. F. Plow and T. V. Byzova (2007). “Mechanisms of integrin-vascular endothelial growth factor receptor cross-activation in angiogenesis.” Circ Res 101(6): 570-80.
Maile, L. A., W. H. Busby, K. Sitko, B. E. Capps, T. Sergent, J. Badley-Clarke and D. R. Clemmons (2006). “Insulin-like growth factor-I signaling in smooth muscle cells is regulated by ligand binding to the 177CYDMKTTC184 sequence of the beta3-subunit of alphaVbeta3.” Mol. Endocrinol. 20(2): 405-13.
Marchetti, F., J. B. Bishop, X. Lowe, W. M. Generoso, J. Hozier, and A. J. Wyrobek, Etoposide induces heritable chromosomal aberrations and aneuploidy during male meiosis in the mouse. Proc. Natl. Acad. Sci. U.S.A., 2001. 98(7): p. 3952-7.
Menendez, J. A., I. Mehmi, and R. Lupu, Heregulin-triggered Her-2/neu signaling enhances nuclear accumulation of p21WAF1/CIP1 and protects breast cancer cells from cisplatin-induced genotoxic damage. Int J Oncol, 2005. 26(3): p. 649-59.
Menendez, J. A., I. Mehmi, and R. Lupu, Trastuzumab in combination with heregulin-activated Her-2 (erbB-2) triggers a receptor-enhanced chemosensitivity effect in the absence of Her-2 overexpression. J Clin Oncol, 2006. 24(23): p. 3735-46.
Meyer, D., and Birchmeier, C. (1995) Nature 378, 386-390.
Meyer, D., Yamaai, T., Garratt, A., Riethmacher-Sonnenberg, E., Kane, D., Theill, L. E., and Birchmeier, C. (1997) Development 124, 3575-3586.
Miyamoto, S., Teramoto, H., Gutkind, J. S., and Yamada, K. M. (1996) J Cell Biol 135, 1633- 1642.
Montero, J. C., Rodriguez-Barrueco, R., Ocana, A., Diaz-Rodriguez, E., Esparis-Ogando, A., and Pandiella, A. (2008) Clin Cancer Res 14, 3237-3241.
Mori, S., Wu, C. Y Yamaji, S., Saegusa, J., Shi, B., Ma, Z., Kuwabara, Y., Lam, K. S., Isseroff, R. R., Takada, Y. K., and Takada, Y. (2008) J Biol Chem 283, 18066-18075.
Muthuswamy, S. K. (2006). “ErbB2 makes beta 4 integrin an accomplice in tumorigenesis.” Cell 126(3): 443-5.
Naidu, R., M. Yadav, S. Nair and M. K. Kutty (1998). “Expression of c-erbB3 protein in primary breast carcinomas.” Br J Cancer 78(10): 1385-90.
Nakano, N., S. Higashiyama, H. Ohmoto, H. Ishiguro, N. Taniguchi and Y. Wada (2004). “The N-terminal region of NTAK/neuregulin-2 isoforms has an inhibitory activity on angiogenesis.” J Biol Chem 279(12): 11465-70.
Olayioye, M. A., R. M. Neve, H. A. Lane and N. E. Hynes (2000). “The ErbB signaling network: receptor heterodimerization in development and cancer.” Embo J 19(13): 3159-67.
Peles, E., Bacus, S. S., Koski, R. A., Lu, H. S., Wen, D., Ogden, S. G., Levy, R. B., and Yarden, Y. (1992) Cell 69, 205-216.
Peng, L., R. Liu, J. Marik, X. Wang, Y. Takada and K. S. Lam (2006). “Combinatorial chemistry identifies high-affinity peptidomimetics against alpha4beta1 integrin for in vivo tumor imaging.” Nature chemical biology 2(7): 381-9.
Puzon-McLaughlin, W., T. Kamata and Y. Takada (2000). “Multiple discontinuous ligand-mimetic antibody binding sites define a ligand binding pocket in integrin alpha(IIb)beta(3).” J Biol Chem 275(11): 7795-802.
Rajkumar, T. and W. J. Gullick (1994). “The type I growth factor receptors in human breast cancer.” Breast Cancer Res Treat 29(1): 3-9.
Riese, D. J., 2nd and D. F. Stern (1998). “Specificity within the EGF family/ErbB receptor family signaling network.” Bioessays 20(1): 41-8.
Roskoski, R., Jr. (2004). “The ErbB/HER receptor protein-tyrosine kinases and cancer.” Biochem Biophys Res Commun 319(1): 1-11.
Russell, K. S., D. F. Stern, P. J. Polverini and J. R. Bender (1999). “Neuregulin activation of ErbB receptors in vascular endothelium leads to angiogenesis.” Am J Physiol 277(6 Pt 2): H2205-11.
Rusnati, M., Tanghetti, E., Dell'Era, P., Gualandris, A., and Presta, M. (1997) Mol Biol Cell 8, 2449-2461.
Saegusa, J., Akakura, N., Wu, C. Y., Hoogland, C., Ma, Z., Lam, K. S., Liu, F. T., Takada, Y. K., and Takada, Y. (2008) J Biol Chem 283, 26107-26115.
Sanner, M. F. (1999) J Mol Graph Model 17, 57-61.
Schmitt, A., Parlapani, E., Gruber, O., Wobrock, T., and Falkai, P. (2008) Eur Arch Psychiatry Clin Neurosci 258 Suppl 5, 35-39.
Shak, S. (1999). “Overview of the trastuzumab (Herceptin) anti-HER2 monoclonal antibody clinical program in HER2-overexpressing metastatic breast cancer. Herceptin Multinational Investigator Study Group.” Semin Oncol 26(4 Suppl 12): 71-7.
Shimaoka, M. and T. A. Springer (2003). “Therapeutic antagonists and conformational regulation of integrin function.” Nat Rev Drug Discov 2(9): 703-16.
Siegel, P. M., E. D. Ryan, R. D. Cardiff and W. J. Muller (1999). “Elevated expression of activated forms of Neu/ErbB-2 and ErbB-3 are involved in the induction of mammary tumors in transgenic mice: implications for human breast cancer.” Embo J 18(8): 2149-64.
Slamon, D. J., G. M. Clark, S. G. Wong, W. J. Levin, A. Ullrich and W. L. McGuire (1987). “Human breast cancer: correlation of relapse and survival with amplification of the HER-2/neu oncogene.” Science 235(4785): 177-82.
Slamon, D. J., W. Godolphin, L. A. Jones, J. A. Holt, S. G. Wong, D. E. Keith, W. J. Levin, S. G. Stuart, J. Udove, A. Ullrich and et al. (1989). “Studies of the HER-2/neu proto-oncogene in human breast and ovarian cancer.” Science 244(4905): 707-12.
Stove, C., V. Stove, L. Derycke, V. Van Marck, M. Mareel and M. Bracke (2003). “The heregulin/human epidermal growth factor receptor as a new growth factor system in melanoma with multiple ways of deregulation.” J Invest Dermatol 121(4): 802-12.
Takada, Y., M. J. Elices, C. Crous and M. E. Hemler (1989). “The primary structure of the a4 subunit of VLA-4: Homology to other integrins and a possible cell-cell adhesion function.” EMBO J. 8: 1361-1368.
Takada, Y. and M. E. Hemler (1989). “The primary structure of the VLA-2/collagen receptor a2 subunit (platelet GP Ia): Homology to other integrins and the presence of possible collagen binding domain.” J. Cell Biol. 109: 397-407.
Takada, Y., C. Huang and M. E. Hemler (1987). “Fibronectin receptor structures within the VLA family of heterodimers.” Nature 326: 607-609.
Takada, Y., T. Kamata, A. Irie, W. Puzon-McLaughlin and X.-P. Zhang (1997). “Structural basis of integrin-mediated signal transduction.” Matrix Biol. 16: 143-151.
Takada, Y., E. Murphy, P. Pil, C. Chen, M. H. Ginsberg and M. E. Hemler (1991). “Molecular cloning and expression of the cDNA for a3 subunit of the human a3b1 (VLA3), an integrin receptor for Fibronectin, laminin and collagen.” J. Cell Biol. 115: 257-266.
Takada, Y., J. L. Strominger and M. E. Hemler (1987). “The VLA family of heterodimers are members of a superfamily of molecules involved in adhesion and embryogenesis.” Proc. Natl. Acad. Sci. 84: 3239-3243.
Takada, Y., Ye, X., and Simon, S. (2007) Genome Biol 8 215.
Takagi, J., T. Kamata, J. Meredith, W. Puzon-McLaughlin and Y. Takada (1997). “Changing ligand specificities of alphavbeta1 and alphavbeta3 integrins by swapping a short diverse sequence of the beta subunit.” J. Biol. Chem. 272(32): 19794-800.
Tan, M., R. Grijalva and D. Yu (1999). “Heregulin betal-activated phosphatidylinositol 3-kinase enhances aggregation of MCF-7 breast cancer cells independent of extracellular signal-regulated kinase.” Cancer Res 59(7): 1620-5.
Tang, C. K., C. Perez, T. Grunt, C. Waibel, C. Cho and R. Lupu (1996). “Involvement of heregulin-beta2 in the acquisition of the hormone-independent phenotype of breast cancer cells.” Cancer Res 56(14): 3350-8.
Tarui, T., N. Akakura, M. Majumdar, N. Andronicos, J. Takagi, A. P. Mazar, K. Bdeir, A. Kuo, S. V. Yarovoi, D. B. Cines and Y. Takada (2006). “Direct interaction of the kringle domain of urokinase-type plasminogen activator (uPA) and integrin alpha v beta 3 induces signal transduction and enhances plasminogen activation.” Thromb Haemost 95(3): 524-34.
Tarui, T., N. Andronicos, R. P. Czekay, A. P. Mazar, K. Bdeir, G. C. Parry, A. Kuo, D. J. Loskutoff, D. B. Cines and Y. Takada (2003). “Critical role of integrin alpha 5 beta 1 in urokinase (uPA)/urokinase receptor (uPAR, CD87) signaling.” J. Biol. Chem. 278(32): 29863-72.
Tarui, T., M. Majumdar, L. A. Miles, W. Ruf and Y. Takada (2002). “Plasmin-induced migration of endothelial cells: A potential target for the anti-angiogenic action of angiostatin.” J. Biol. Chem. 277: 33564-33570.
Tarui, T., A. P. Mazar, D. B. Cines and Y. Takada (2001). “Urokinase-type plasminogen activator receptor (CD87) is a ligand for integrins and mediates cell-cell interaction.” J. Biol. Chem. 276(6): 3983-90.
Tarui, T., L. A. Miles and Y. Takada (2001). “Specific interaction of angiostatin with integrin {alpha}v{beta}3 in endothelial cells.” J. Biol. Chem. 276: 39562-39568.
Traxler, P., P. R. Allegrini, R. Brandt, J. Brueggen, R. Cozens, D. Fabbro, K. Grosios, H. A. Lane, P. McSheehy, J. Mestan, T. Meyer, C. Tang, M. Wartmann, J. Wood and G. Caravatti (2004). “AEE788: a dual family epidermal growth factor receptor/ErbB2 and vascular endothelial growth factor receptor tyrosine kinase inhibitor with antitumor and antiangiogenic activity.” Cancer research 64(14): 4931-41.
Tsai, M. S., L. A. Shamon-Taylor, I. Mehmi, C. K. Tang and R. Lupu (2003). “Blockage of heregulin expression inhibits tumorigenicity and metastasis of breast cancer.” Oncogene 22(5): 761-8.
Vellon, L., J. A. Menendez and R. Lupu (2005). “AlphaVbeta3 integrin regulates heregulin (HRG)-induced cell proliferation and survival in breast cancer.” Oncogene 24(23): 3759-73.
Wang, W., and Malcolm, B. A. (1999) Biotechniques 26, 680-682.
White, D. E., N. A. Kurpios, D. Zuo, J. A. Hassell, S. Blaess, U. Mueller, and W. J. Muller, Targeted disruption of beta1-integrin in a transgenic mouse model of human breast cancer reveals an essential role in mammary tumor induction. Cancer Cell, 2004. 6(2): p. 159-70.
Wu, P. L., S. C. Lee, C. C. Chuang, S. Mori, N. Akakura, W. G. Wu and Y. Takada (2006). “Non-cytotoxic cobra cardiotoxin A5 binds to integrin alpha vbeta 3 and inhibits bone resorption: Identification of cardiotoxins as non-RGD integrin-binding proteins of the Ly-6 family.” J Biol Chem.
Xiong, J. P., T. Stehle, R. Zhang, A. Joachimiak, M. Frech, S. L. Goodman and M. A. Arnaout (2002). “Crystal structure of the extracellular segment of integrin alpha Vbeta3 in complex with an Arg-Gly-Asp ligand.” Science 296(5565): 151-5.
Xiong, S., R. Grijalva, L. Zhang, N. T. Nguyen, P. W. Pisters, R. E. Pollock and D. Yu (2001). “Up-regulation of vascular endothelial growth factor in breast cancer cells by the heregulin-beta1-activated p38 signaling pathway enhances endothelial cell migration.” Cancer Res 61(4): 1727-32.
Xu, F. J., S. Stack, C. Boyer, K. O'Briant, R. Whitaker, G. B. Mills, Y. H. Yu and R. C. Bast, Jr. (1997). “Heregulin and agonistic anti-p185(c-erbB2) antibodies inhibit proliferation but increase invasiveness of breast cancer cells that overexpress p185(c-erbB2): increased invasiveness may contribute to poor prognosis.” Clin Cancer Res 3(9): 1629-34.
Yen, L., N. Benlimame, Z. R. Nie, D. Xiao, T. Wang, A. E. Al Moustafa, H. Esumi, J. Milanini, N. E. Hynes, G. Pages and M. A. Alaoui-Jamali (2002). “Differential regulation of tumor angiogenesis by distinct ErbB homo- and heterodimers.” Mol Biol Cell 13(11): 4029-44.
Yen, L., X. L. You, A. E. Al Moustafa, G. Batist, N. E. Hynes, S. Mader, S. Meloche and M. A. Alaoui-Jamali (2000). “Heregulin selectively upregulates vascular endothelial growth factor secretion in cancer cells and stimulates angiogenesis.” Oncogene 19(31): 3460-9.
Yumoto, N., X. Yu and M. Hatakeyama (2006). “Expression of the ErbB4 receptor causes reversal regulation of PP2A in the Shc signal transduction pathway in human cancer cells.” Mol Cell Biochem 285(1-2): 165-71.
Zamarron, C., M. Ginsberg and E. Plow (1990). “Monoclonal antibodies specific for a conformationally altered state of fibrinogen.” Thrombosis & Haemostasis. 64: 41-46.
Zhang, X. P., T. Kamata, K. Yokoyama, W. Puzon-McLaughlin and Y. Takada (1998). “Specific interaction of the recombinant disintegrin-like domain of MDC-15 (metargidin, ADAM-15) with integrin alphavbeta3.” J Biol Chem 273(13): 7345-50.
Zhang, X. P., T. Kamata, K. Yokoyama, W. Puzon-McLaughlin and Y. Takada (1998). “Specific interaction of the recombinant disintegrin-like domain of MDC- 15 (metargidin, ADAM-15) with integrin alphavbeta3.” J. Biol. Chem. 273(13): 7345-50.
Zhang, D., Sliwkowski, M. X., Mark, M., Frantz, G., Akita, R., Sun, Y., Hillan, K., Crowley, C., Brush, J., and Godowski, P. J. (1997) Proc Natl Acad Sci USA 94, 9562-9567.
Zhao, Y. Y., O. Feron, C. Dessy, X. Han, M. A. Marchionni, and R. A. Kelly, Neuregulin signaling in the heart. Dynamic targeting of erbB4 to caveolar microdomains in cardiac myocytes. Circ Res, 1999. 84(12): p. 1380-7.
Zhao, Z. G., J. S. Im, K. S. Lam and D. F. Lake (1999). “Site-specific modification of a single-chain antibody using a novel glyoxylyl-based labeling reagent.” Bioconjug Chem 10(3): 424-30.
Zhou, X., F. R. Murphy, N. Gehdu, J. Zhang, J. P. Iredale, and R. C. Benyon, Engagement of alphavbeta3 integrin regulates proliferation and apoptosis of hepatic stellate cells. J Biol Chem, 2004. 279(23): p. 23996-4006.

SEQUENCE LISTING

SEQ ID NO: 1
Amino acid sequence of an integrin-binding site within human neuregulin
1 EGF-like region:

| | |
|---|---|
| 1 | VKCAEKEKTF CVNGGECF |

SEQ ID NO: 2
Amino acid sequence of an integrin-binding site (human neuregulin 1
EGF-like region and a portion of the α domain):

| | |
|---|---|
| 1 | HLVKCAEKEK TFCVNGGECF MVKDLSNPSR YLCKCQPGFT GARCT |

SEQ ID NO: 3
Amino acid sequence of an integrin-binding site (human neuregulin 1 EGF-like region
and a portion of the α domain, residues 175-222 of SEQ ID NO: 4, short version):

| | |
|---|---|
| 1 | GTSHLVKCAE KEKTFCVNGG ECFMVKDLSN PSRYLCKCQP GFTGARCT |

SEQ ID NO: 4
Amino acid sequence of human neuregulin 1α (GenBank Accession No. AAA58638):

| | |
|---|---|
| 1 | MSERKEGRGK GKGKKKERGS GKKPESAAGS QSPALPPRLK EMKSQESAAG SKLVLRCETS |
| 61 | SEYSSLRFKW FKNGNELNRK NKPQNIKIQK KPGKSELRIN KASLADSGEY MCKVISKLGN |
| 121 | DSASANITIV ESNEIITGMP ASTEGAYVSS ESPIRISVST EGANTSSSTS TSTTGTSHLV |
| 181 | KCAEKEKTFC VNGGECFMVK DLSNPSRYLC KCQPGFTGAR CTENVPMKVQ NQEKAEELYQ |
| 241 | KRVLTITGIC IALLVVGIMC VVAYCKTKKQ RKKLHDRLRQ SLRSERNNMM NIANGPHHPN |
| 301 | PPPENVQLVN QYVSKNVISS EHIVEREAET SFSTSHYTST AHHSTTVTQT PSHSWSNGHT |
| 361 | ESILSESHSV IVMSSVENSR HSSPTGGPRG RLNGTGGPRE CNSFLRHARE TPDSYRDSPH |
| 421 | SERYVSAMTT PARMSPVDFH TPSSPKSPPS EMSPPVSSMT VSMPSMAVSP FMEEERPLLL |
| 481 | VTPPRLREKK FDHHPQQFSS FHHNPAHDSN SLPASPLRIV EDEEYETTQE YEPAQEPVKK |
| 541 | LANSRRAKRT KPNGHIANRL EVDSNTSSQS SNSESETEDE RVGEDTPFLG IQNPLAASLE |
| 601 | ATPAFRLADS RTNPAGRFST QEEIQARLSS VIANQDPIAV |

SEQ ID NO: 5
Amino acid sequence of an integrin-binding site within human neuregulin
(residues 175-241 of SEQ ID NO: 4, long version)

| | |
|---|---|
| 1 | GTSHLVKCAEKEKTFCVNGGECFMVKDLSNPSRYLCKCQPGFTGARCTENVPMKVQNQEKAEELYQK |

SEQ ID NO: 6 (3KE, long version)
Amino acid sequence of 3KE mutant, long version

| | |
|---|---|
| 1 | GTSHLVeCAEeEeTFCVNGGECFMVKDLSNPSRYLCKCQPGFTGARCTENVPMKVQNQEKAEELYQK |

(mutations are shown in a lower case e)
SEQ ID NO: 7 (2KE, long version)
Amino acid sequence of 2KE mutant, long version

| | |
|---|---|
| 1 | GTSHLVKCAEeEeTFCVNGGECFMVKDLSNPSRYLCKCQPGFTGARCTENVPMKVQNQEKAEELYQK |

(mutations are shown in a lower case e)
SEQ ID NO: 8
Human neuregulin 1β:

| | |
|---|---|
| 1 | MSERKEGRGK GKGKKKERGS GKKPESAAGS QSPALPPRLK EMKSQESAAG SKLVLRCETS |
| 61 | SEYSSLRFKW FKNGNELNRK NKPQNIKIQK KPGKSELRIN KASLADSGEY MCKVISKLGN |
| 121 | DSASANITIV ESNEIITGMP ASTEGAYVSS ESPIRISVST EGANTSSSTS TSTTGTSHLV |
| 181 | KCAEKEKTFC VNGGECFMVK DLSNPSRYLC KCPNEFTGDR CQNYVMASFY KHLGIEFMEA |
| 241 | EELYQKRVLT ITGICIALLV VGIMCVVAYC KTKKQRKKLH DRLRQSLRSE RNNMMNIANG |
| 301 | PHHPNPPPEN VQLVNQYVSK NVISSEHIVE REAETSFSTS HYTSTAHHST TVTQTPSHSW |
| 361 | SNGHTESILS ESHSVIVMSS VENSRHSSPT GGPRGRLNGT GGPRECNSFL RHARETPDSY |
| 421 | RDSPHSERYV SAMTTPARMS PVDFHTPSSP KSPPSEMSPP VSSMTVSMPS MAVSPFMEEE |
| 481 | RPLLLVTPPR LREKKFDHHP QQFSSFHHNP AHDSNSLPAS PLRIVEDEEY ETTQEYEPAQ |
| 541 | EPVKKLANSR RAKRTKPNGH IANRLEVDSN TSSQSSNSES ETEDERVGED TPFLGIQNPL |
| 601 | AASLEATPAF RLADSRTNPA GRFSTQEEIQ ARLSSVIANQ DPIAV |

SEQ ID NO: 9
NRG1β long, residues 175-246 of SEQ ID NO: 8, long version
GTSHLVKCAEKEKTFCVNGGECFMVKDLSNPSRYLCKCQPGFTGARCTENVPMKVQNQEKAEELYQK
SEQ ID NO: 10
Murine NRG1β fragment
TSHLIKCAEKEKTFCVNGGECFMVKDLSNPSRYLCKCPNEFTGDRCQNYVMASFYKHLGIEFMEAEELYQK
SEQ ID NO: 11 (3KE, short version)
Amino acid sequence of 3KE mutant, short version

| | |
|---|---|
| 1 | GTSHLVeCAEeEeTFCVNGGECFMVKDLSNPSRYLCKCQPGFTGARCT |

(mutations are shown in a lower case e)
SEQ ID NO: 12 (2KE, short version)
Amino acid sequence of 2KE mutant, short version

| | |
|---|---|
| 1 | GTSHLVKCAEeEeTFCVNGGECFMVKDLSNPSRYLCKCQPGFTGARCT |

(mutations are shown in a lower case e)

1. A method for inhibiting proliferation of a cell, comprising the step of contacting the cell with an effective amount of an inhibitor of neuregulin-integrin binding.

2. The method of claim 1, wherein the neuregulin is neuregulin 1α (NRG1α) or neuregulin 1β (NRG1β).

3. The method of claim 1, wherein the integrin is αvβ3, α6β4, α6β1 or α9β1.

4. The method of claim 1, wherein the inhibitor is a polypeptide comprising a core amino acid sequence corresponding to residues 197-241 of SEQ ID NO:4 or residues 197-246 of SEQ ID NO:8, wherein, if the polypeptide comprises an additional amino acid sequence, the additional amino acid sequence does not comprise a sequence corresponding to residues 181-187 of SEQ ID NO:4 or 8, and wherein the polypeptide inhibits neuregulin-integrin binding.

5. The method of claim 4, wherein the additional amino acid sequence, if any, is located at the N-terminus of the core amino acid sequence.

6. The method of claim 4, wherein the core amino acid sequence corresponds to residues 190-241 of SEQ ID NO:4 or residues 190-246 of SEQ ID NO:8.

7. The method of claim 4, wherein the polypeptide comprises the amino acid sequence of residues 175-241 of SEQ ID NO:4 or 175-246 of SEQ ID NO:8, with at least one of three Lys residues at positions 181, 185, and 187 of SEQ ID NO:4 or SEQ ID NO:8 substituted or deleted, such that the polypeptide inhibits neuregulin-integrin binding.

8. The method of claim 7, wherein at least two of the three Lys residues at positions 181, 185, and 187 of SEQ ID NO:4 or SEQ ID NO:8 are substituted.

9. The method of claim 7, wherein the inhibitor is a polypeptide comprising the amino acid sequence of SEQ ID NO:6.

10. The method of claim 7, wherein the inhibitor is a polypeptide comprising the amino acid sequence of SEQ ID NO:7.

11. The method of claim 4, the polypeptide further comprises a heterologous amino acid sequence.

12. The method of claim 11, wherein the heterologous amino acid sequence is glutathione S-transferase (GST) sequence.

13. The method of claim 1, wherein the inhibitor is anti-β3 antibody 7E3.

14. The method of claim 1, wherein the inhibitor is cyclic RGDfV.

15. The method of claim 1, wherein the cell is within a patient's body.

16. The method of claim 1, wherein the contacting step is performed by subcutaneous, intramuscular, intravenous, intraperitoneal, or intratumor injection.

17. The method of claim 4, wherein the effective amount is 1 μg/kg to 1 mg/kg body weight.

18. A method for identifying an inhibitor of neuregulin-integrin binding, comprising the steps of
(1) contacting an integrin and a polypeptide comprising an integrin-binding sequence of a neuregulin, in the presence of a test compound, under conditions permissible for neuregulin-integrin binding; and
(2) detecting the level of polypeptide-integrin binding, wherein a decrease in the level of binding when compared with the level of binding in the absence of the test compound indicates the compound as an inhibitor of neuregulin-integrin binding.

19. The method of claim 18, wherein the integrin-binding sequence comprises the amino acid sequence of residues 181-187 of SEQ ID NO:4 or 8.

20. The method of claim 18, wherein the neuregulin is neuregulin 1α (NRG1α) or neuregulin 1β (NRG1β).

21. The method of claim 18, wherein the integrin is αvβ3, α6β4, α6β1 or α9β1.

22. The method of claim 19, wherein the polypeptide comprises SEQ ID NO: 1, 2, 3, 4, 5, 8, or 9.

23. The method of claim 19, wherein the polypeptide further comprises a heterologous amino acid sequence.

24. The method of claim 23, wherein the heterologous amino acid sequence is glutathione S-transferase (GST) sequence.

25. The method of claim 19, wherein the integrin is expressed on a cell surface.

26. An isolated polypeptide comprising a core amino acid sequence corresponding to residues 197-241 of SEQ ID NO:4 or residues 197-246 of SEQ ID NO:8, wherein, if the polypeptide comprises an additional amino acid sequence, the additional amino acid sequence does not comprise a sequence corresponding to residues 181-187 of SEQ ID NO:4 or 8, and wherein the polypeptide inhibits neuregulin-integrin binding.

27. The isolated polypeptide of claim 26, wherein the additional amino acid sequence, if any, is located at the N-terminus of the core amino acid sequence.

28. The polypeptide of claim 26, wherein the core amino acid sequence corresponds to residues 190-241 of SEQ ID NO:4 or residues 190-246 of SEQ ID NO:8.

29. The polypeptide of claim 26, wherein the integrin is αvβ3, α6β4, α6β1 or α9β1.

30. The polypeptide of claim 26, wherein at least two of the three Lys residues at positions 181, 185, and 187 of SEQ ID NO:4 or SEQ ID NO:8 are substituted.

31. The polypeptide of claim 26, wherein the Lys residues at positions 185 and 187 but not 181 are substituted.

32. The polypeptide of claim 26, wherein the Lys residues at positions 181, 185, and 187 are substituted.

33. The polypeptide of claim 30, wherein each of the substituted Lys residues is substituted with a Glu residue.

34. A method for inhibiting proliferation of a cell, comprising the step of transfecting the cell with a nucleic acid encoding the polypeptide of claim 26.

35. A composition comprising the polypeptide of claim 26 and a pharmaceutically acceptable excipient.

36. The composition of claim 19, wherein the polypeptide comprises the amino acid sequence of SEQ ID NO:6 or 7.

37. The composition of claim 19, wherein the polypeptide further comprises a heterologous amino acid sequence.

38. The composition of claim 37, wherein the heterologous amino acid sequence is glutathione S-transferase (GST) sequence.

39. An isolated nucleic acid encoding the polypeptide of claim 26.

40. A recombinant expression cassette comprising the nucleic acid of claim 39.

41. An isolated host cell comprising the expression cassette of claim 40.

42. A composition comprising the nucleic acid of claim 39 or the expression cassette of claim 40, and a pharmaceutically acceptable excipient.

43. A kit for inhibiting proliferation of a cell, comprising the composition of claim 35.

44. A kit for identifying an inhibitor of neuregulin-integrin binding, comprising an integrin and a polypeptide comprising an integrin-binding sequence of a neuregulin.

45. A kit for inhibiting proliferation of a cell, comprising the composition of claim 42.
